(19) United States
(12) Patent Application Publication
(10) Pub. No.: US 2026/0237457 A1
(43) Pub. Date: Aug. 13, 2026

(54) Method, Apparatus and Computer Program for Reliably Reporting Uncertain DNA Evidence

(71) Applicant: Mark w. Perlin, Pittsburgh, PA (US)

(72) Inventor: Mark w. Perlin, Pittsburgh, PA (US)

(21) Appl. No.: 19/532,835

(22) Filed: Feb. 6, 2026

Related U.S. Application Data
(60) Provisional application No. 63/756,368, filed on Feb. 10, 2025.

(51) Int. Cl. G16B 20/00; C12Q 1/6886

(52) U.S. Cl. CPC G16B 20/00; C12Q 1/6886

(57) ABSTRACT

A method of convicting a person guilty of a crime having the step of collecting evidence from a scene where a crime has occurred that contains DNA from a specific person. There is the step of interpreting DNA data to infer a probabilistic evidence genotype. The evidence genotype is compared with the person's genotype to produce an inclusionary score. The score, together with a component of a package, is presented in a legal proceeding. The person of the crime is convicted, based on a score presentation, and incarcerated. A method of acquitting a person not guilty of a crime. A non-transitory readable storage medium and an apparatus for convicting or acquitting a person.

### CROSS-REFERENCE TO RELATED APPLICATIONS

This is a nonprovisional application of U.S. provisional application Ser. No. 63/756,368 filed Feb. 10, 2025, incorporated by reference herein.

### FIELD OF THE INVENTION

The present invention is related to a method of convicting a person guilty or of acquitting a person not guilty of a crime based on an inclusionary score or an exclusionary score, respectively. More specifically, the present invention is related to a method of convicting a person guilty or of acquitting a person not guilty of a crime based on an inclusionary score or an exclusionary score, respectively, using a probabilistic evidence genotype.

### BACKGROUND OF THE INVENTION

This section is intended to introduce the reader to various aspects of the art that may be related to various aspects of the present invention. The following discussion is intended to provide information to facilitate a better understanding of the present invention. Accordingly, it should be understood that statements in the following discussion are to be read in this light, and not as admissions of prior art.

Informative DNA evidence often yields uninformative results. Most crime laboratories apply binary threshold criteria that discard DNA data and inhibit reporting. The information loss is more common with complex evidence having multiple contributors or little DNA. Probative inculpatory and exculpatory evidence is lost to criminal justice.

Probability provides a non-binary way to accurately represent genotype uncertainty. Bayesian models scrutinize evidence data to transform prior to posterior genotype probability. A likelihood ratio (LR) gives the evidential change for a reference. A LR distribution charts the LR values at every reference, for Noncontributor or Contributor categories. The previous convolution invention efficiently constructed dense LR distributions directly from an evidence genotype, without computing any LRs. A distribution can determine LR error rates.

The instant invention applies LR distributions and error rates to reliably report LR results from uncertain genotypes. The specification presents the symmetry between inclusionary and exclusionary match statistics, which have equal mathematical standing. It dissects two low-LR cases, one a criminal conviction and the other an exoneration, illustrating LR reporting, validation, and error rate. It shows how thresholds reduce DNA information and when they are unnecessary. It explains why casework field data can be used for genotyping validation.

A novel application of receiver operating characteristic (ROC) analysis to a genotype's Noncontributor and Contributor LR score distributions generates a continuous ROC curve. The area under the curve (AUC) is an objective measure of discrimination accuracy. The invention uses the AUC to assess genotype accuracy, compare genotyping methods, and understand how data thresholds reduce DNA match information.

Probabilistic genotyping examines DNA data to form an uncertain genotype. This single genotype outcome contains everything needed to build its Noncontributor and Contributor LR distributions, calculate error rates, conduct ROC analysis, and measure accuracy. The genotype provides a complete statistical framework, before ever comparing with a reference.

### SUMMARY OF THE INVENTION

The present invention pertains to a method of convicting a person guilty of a crime. The method comprises the step of a. collecting evidence from a scene where a crime has occurred that contains DNA from a specific person. There is the step of b. processing the evidence to produce DNA data. There is the step of c. interpreting the data to infer a probabilistic evidence genotype. There is the step of d. deriving from the evidence genotype a first score distribution that describes the frequency of scores from reference genotypes of people who did not contribute their DNA to the evidence. There is the step of e. deriving a second score distribution from the evidence genotype that describes the frequency of scores from reference genotypes that are probabilistically similar to the genotype of the person who contributed their DNA to the evidence. There is the step of constructing a curve from pairs of error rates at a plurality of scores, where each error rate pair comes from the first and second distributions evaluated at the score. There is the step of g. finding an area under the curve. There is the step of h. using the area as a measure of the score's ability to accurately discriminate between people who did or did not leave their DNA in the evidence. There is the step of i. organizing the genotype's score distribution pair, curve, and area into a discrimination package. There is the step of j. comparing the evidence genotype with the person's genotype to produce an inclusionary score. There is the step of k. presenting the score, together with a component of the package, in a legal proceeding. There is the step of l. convicting the person of the crime, based on the score presentation. There is the step of m. incarcerating the person.

The present invention pertains to a method of acquitting a person not guilty of a crime. The method comprises the steps a. collecting evidence from a scene where a crime has occurred that does not contain DNA from a specific person. There is the step of b. processing the evidence to produce DNA data. There is the step of c. interpreting the data to infer a probabilistic evidence genotype. There is the step of d. deriving from the evidence genotype a first score distribution that describes the frequency of scores from reference genotypes of people who did not contribute their DNA to the evidence. There is the step of e. deriving a second score distribution from the evidence genotype that describes the frequency of scores from reference genotypes that are probabilistically similar to the genotype of someone who is not the person and contributed their DNA to the evidence. There is the step of f. constructing a curve from pairs of error rates at a plurality of scores, where each error rate pair comes from the first and second distributions evaluated at the score. There is the step of g. finding an area under the curve. There is the step of h. using the area as a measure of the score's ability to accurately discriminate between people who did or did not leave their DNA in the evidence. There is the step of i. organizing the genotype's score distribution pair, curve, and area into a discrimination package. There is the step of j. comparing the evidence genotype with the person's genotype to produce an exclusionary score. There is the step of k. presenting the score, together with a component of the package, in a legal proceeding. There is the step of l. acquitting or exonerating the person of the crime, based on the score presentation. There is the step of m. releasing the person.

The present invention pertains to a non-transitory readable storage medium includes a computer program stored on the storage medium for convicting a person guilty of a crime, as shown in FIG. 22. The computer program has the computer-generated steps of processing evidence from a scene where a crime has occurred that contains DNA from a specific person to produce DNA data. There is the step of interpreting the data to infer a probabilistic evidence genotype. There is the step of deriving from the evidence genotype a first score distribution that describes the frequency of scores from reference genotypes of people who did not contribute their DNA to the evidence. There is the step of deriving a second score distribution from the evidence genotype that describes the frequency of scores from reference genotypes that are probabilistically similar to the genotype of the person who contributed their DNA to the evidence. There is the step of constructing a curve from pairs of error rates at a plurality of scores, where each error rate pair comes from the first and second distributions evaluated at the score. There is the step of finding an area under the curve. There is the step of using the area as a measure of the score's ability to accurately discriminate between people who did or did not leave their DNA in the evidence. There is the step of organizing the genotype's score distribution pair, curve, and area into a discrimination package. There is the step of comparing the evidence genotype with the person's genotype to produce an inclusionary score; wherein the score is presented, together with a component of the package, in a legal proceeding causing conviction of the person of the crime, based on the score presentation, and incarcerating the person.

An apparatus for convicting a person guilty of a crime. The apparatus comprises a computer. The apparatus comprises a non-transitory readable storage medium in communication with the computer. The non-transitory readable storage medium includes a computer program stored on the storage medium. The computer program has the computer-generated steps of processing evidence from a scene where a crime has occurred that contains DNA from a specific person to produce DNA data. There is the step of interpreting the data to infer a probabilistic evidence genotype. There is the step of deriving from the evidence genotype a first score distribution that describes the frequency of scores from reference genotypes of people who did not contribute their DNA to the evidence. There is the step of deriving a second score distribution from the evidence genotype that describes the frequency of scores from reference genotypes that are probabilistically similar to the genotype of the person who contributed their DNA to the evidence. There is the step of constructing a curve from pairs of error rates at a plurality of scores, where each error rate pair comes from the first and second distributions evaluated at the score. There is the step of finding an area under the curve. There is the step of using the area as a measure of the score's ability to accurately discriminate between people who did or did not leave their DNA in the evidence. There is the step of organizing the genotype's score distribution pair, curve, and area into a discrimination package. There is the step of comparing the evidence genotype with the person's genotype to produce an inclusionary score; wherein the score is presented, together with a component of the package, in a legal proceeding causing conviction of the person of the crime, based on the score presentation, and incarcerating the person.

### BRIEF DESCRIPTION OF THE DRAWINGS

FIG. 1. Non-contributor distribution. (Cumulative) An uncertain genotype's CDF for non-contributor random variable X shows cumulative probability (y-axis) relative to logarithmic match strength (x-axis). (Probability) The corresponding PMF gives the probability. (Reproduced from Heliyon with permission.)

FIG. 2. Contributor distribution. (Cumulative) An uncertain genotype's CDF for contributor random variable Y shows cumulative probability (y-axis) relative to logarithmic match strength (x-axis). (Probability) The corresponding PMF gives the probability. (Reproduced from Heliyon with permission.)

FIG. 3. Distribution tail probability. (Noncontributor) The right tail probability at x (red) gives the false positive rate (FPR), while the left area is the true negative rate (TNR). (Contributor) The left tail probability at x (blue) gives the false negative rate (FNR), while the right area is the true positive rate (TPR).

FIG. 4. Paired distribution overlap. The paired Noncontributor X (red) and Contributor Y (blue) log(LR) score distributions show increasing overlap. The curve pairs proceed from (A) no overlap, to (B) some separation and (C) more separation, ending at (D) complete separation.

FIG. 5. Positive tail probabilities. At a log(LR) score value x, the Contributor distribution's TPR right tail probability (blue) and Noncontributor FPR right tail (red) are shown as areas under the probability mass function curve.

FIG. 6. ROC curves. The ROC x-axis is 1-specificity(x), and its y-axis is sensitivity(x). An ROC curve is a parametric function (FPR(x), TPR(x)) of x=log(LR). The four ROC curves (A, B, C, D) shown correspond to the degrees of distribution pair overlap shown in FIG. 4.

FIG. 7. Inclusionary distribution pair. A Noncontributor (left blue curve) and Contributor (right curve) distribution are shown, developed from a genotype in Johnson. The low inclusionary log(LR) match statistic is indicated (green arrow).

FIG. 8. Inclusionary composite distributions. Composite evidence Noncontributor (left red curve) and Contributor (right magenta) LR distributions assembled from 95 low inclusionary match statistic genotypes.

FIG. 9. Inclusionary ER-LR scatterplot. The log-log scatterplot shows ER as a function of LR for 95 low inclusionary LR match statistics. Each blue dot represents a (log(LR), log(ER)) pair for the FPR ER of the LR. The blue line shows ER=1/LR.

FIG. 10. Exclusionary distribution pair. A Noncontributor (left blue curve) and Contributor (right curve) distribution are shown, developed from a genotype in Robinson. The low exclusionary log(LR) match statistic is indicated (green arrow).

FIG. 11. Exclusionary composite distributions. Composite evidence Noncontributor (left red curve) and Contributor (right magenta) LR distributions assembled from 85 low exclusionary match statistic genotypes.

FIG. 12. Exclusionary ER-LR scatterplot. The log-log scatterplot shows ER as a function of LR for 85 low exclusionary LR match statistics. Each red dot represents a (log(LR), log(ER)) pair for the FNR ER of the LR. The red line shows ER=LR.

FIG. 13. TrueAllele Sandoval distributions. TrueAllele Noncontributor (left blue) and Contributor (right) distributions for the exclusionary genotype in Sandoval. The defendant's exclusionary log(LR) match statistic is shown (green arrow).

FIG. 14. STRmix Sandoval distributions. STRmix Noncontributor (left blue) and Contributor (right) distributions for the exclusionary genotype in Sandoval. The defendant's exclusionary log(LR) match statistic is shown (green arrow).

FIG. 15. Sandoval ROC curves. The TrueAllele (blue) and STRmix (red) ROC curves correspond to the degrees of distribution pair overlap shown in FIGS. 13 (TrueAllele) and 14 (STRmix).

FIG. 16. Sampled Noncontributor histogram. A histogram shows empirical log(LR) Noncontributor distributions for 101 evidence genotype comparisons relative to 10,000 randomly generated references. There are a million data points for each of the three ethnic populations. (Reproduced from PLOS ONE with permission.)

FIG. 17. Exact LR distributions. The composite Noncontributor (left red) and Contributor (right magenta) distributions constructed by convolution from 101 validation genotypes.

FIG. 18. Mills LR distributions. Noncontributor (left blue curve) and Contributor (right curve) LR distributions are shown for (A) gun and (B) magazine evidence. The defendant's exclusionary match statistic (green arrow) is shown for each item, along with seven weaker exclusionary validation statistics (red arrows).

FIG. 19. Burton LR distributions. Noncontributor (left blue curve) and Contributor (right curve) LR distributions are shown for rape kit evidence. The defendant's inclusionary match statistic (rightmost green arrow) is shown, along with twelve weaker database search statistics (other green arrows).

FIG. 20. Next generation sequencing. Composite Noncontributor (red) and Contributor (blue) LR distributions for five NGS genotype sets grouped by contributor genotype mixture weight. (A) 0-20% MW, (b) 21-40% MW, (c) 41-60% MW, (d) 61-80% MW, and (e) 81-100% MW.

FIG. 21. Medical diagnosis. The LR Noncontributor distribution (blue curve) and false positive rate (green dot) for a Stable Angina chest pain diagnosis. LR and ER statements are shown.

FIG. 22. A block diagram of a non-transitory readable storage medium and an apparatus for convicting a person guilty of a crime

### DETAILED DESCRIPTION OF THE INVENTION

### 1. Introduction

The present invention pertains to a method of convicting a person guilty of a crime. The method comprises the step of a. collecting evidence from a scene where a crime has occurred that contains DNA from a specific person. There is the step of b. processing the evidence to produce DNA data. There is the step of c. interpreting the data to infer a probabilistic evidence genotype. There is the step of d. deriving from the evidence genotype a first score distribution that describes the frequency of scores from reference genotypes of people who did not contribute their DNA to the evidence. There is the step of e. deriving a second score distribution from the evidence genotype that describes the frequency of scores from reference genotypes that are probabilistically similar to the genotype of the person who contributed their DNA to the evidence. There is the step of f. constructing a curve from pairs of error rates at a plurality of scores, where each error rate pair comes from the first and second distributions evaluated at the score. There is the step of g. finding an area under the curve. There is the step of h. using the area as a measure of the score's ability to accurately discriminate between people who did or did not leave their DNA in the evidence. There is the step of i. organizing the genotype's score distribution pair, curve, and area into a discrimination package. There is the step of j. comparing the evidence genotype with the person's genotype to produce an inclusionary score. There is the step of k. presenting the score, together with a component of the package, in a legal proceeding. There is the step of l. convicting the person of the crime, based on the score presentation. There is the step of m. incarcerating the person.

Before the step of presenting in a legal proceeding, there may be the additional step of using the score and a component of the package to persuade a judge to admit the DNA evidence. The score may be a likelihood ratio. Only one genotype inferred from the evidence data may be used in deriving the first and second score distributions. Steps a through i may be completed before comparing the evidence genotype with a reference genotype.

The present invention pertains to a method of acquitting a person not guilty of a crime. The method comprises the steps a. collecting evidence from a scene where a crime has occurred that does not contain DNA from a specific person. There is the step of b. processing the evidence to produce DNA data. There is the step of c. interpreting the data to infer a probabilistic evidence genotype. There is the step of d. deriving from the evidence genotype a first score distribution that describes the frequency of scores from reference genotypes of people who did not contribute their DNA to the evidence. There is the step of e. deriving a second score distribution from the evidence genotype that describes the frequency of scores from reference genotypes that are probabilistically similar to the genotype of someone who is not the person and contributed their DNA to the evidence. There is the step of f. constructing a curve from pairs of error rates at a plurality of scores, where each error rate pair comes from the first and second distributions evaluated at the score. There is the step of g. finding an area under the curve. There is the step of h. using the area as a measure of the score's ability to accurately discriminate between people who did or did not leave their DNA in the evidence. There is the step of i. organizing the genotype's score distribution pair, curve, and area into a discrimination package. There is the step of j. comparing the evidence genotype with the person's genotype to produce an exclusionary score. There is the step of k. presenting the score, together with a component of the package, in a legal proceeding. There is the step of l. acquitting or exonerating the person of the crime, based on the score presentation. There is the step of m. releasing the person.

Before the step of presenting in a legal proceeding, there may be the additional step of using the score and a component of the package to persuade a judge to admit the DNA evidence. The score may be a likelihood ratio. Only one genotype inferred from the evidence data may be used in deriving the first and second score distributions. Steps a through i may be completed before comparing the evidence genotype with a reference genotype.

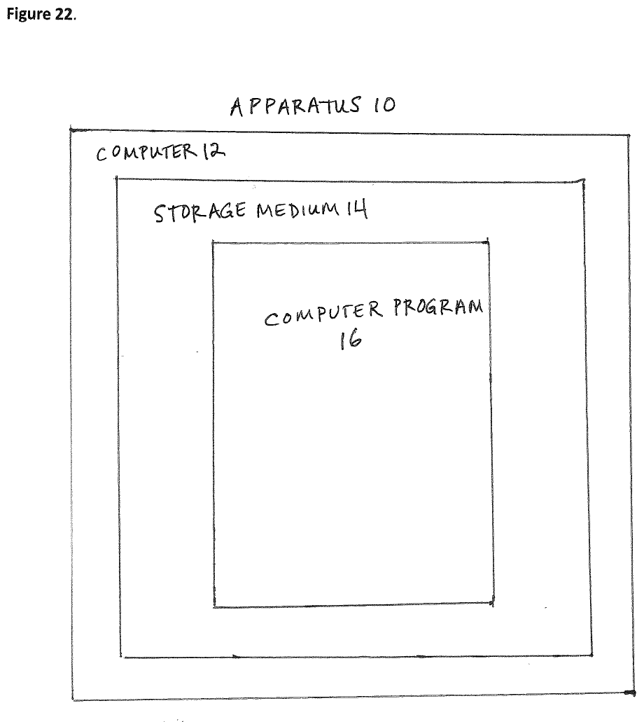

The present invention pertains to a non-transitory readable storage medium 14 includes a computer 12 program stored on the storage medium 14 for convicting a person guilty of a crime, as shown in FIG. 22. The computer 12 program has the computer-generated steps of processing evidence from a scene where a crime has occurred that contains DNA from a specific person to produce DNA data. There is the step of interpreting the data to infer a probabilistic evidence genotype. There is the step of deriving from the evidence genotype a first score distribution that describes the frequency of scores from reference genotypes of people who did not contribute their DNA to the evidence. There is the step of deriving a second score distribution from the evidence genotype that describes the frequency of scores from reference genotypes that are probabilistically similar to the genotype of the person who contributed their DNA to the evidence. There is the step of constructing a curve from pairs of error rates at a plurality of scores, where each error rate pair comes from the first and second distributions evaluated at the score. There is the step of finding an area under the curve. There is the step of using the area as a measure of the score's ability to accurately discriminate between people who did or did not leave their DNA in the evidence. There is the step of organizing the genotype's score distribution pair, curve, and area into a discrimination package. There is the step of comparing the evidence genotype with the person's genotype to produce an inclusionary score; wherein the score is presented, together with a component of the package, in a legal proceeding causing conviction of the person of the crime, based on the score presentation, and incarcerating the person. This can also be used to acquit a person.

An apparatus 10 for convicting a person guilty of a crime, as shown in FIG. 22. The apparatus 10 comprises a computer 12. The apparatus 10 comprises a non-transitory readable storage medium 14 in communication with the computer 12. The non-transitory readable storage medium 14 includes a computer program 16 stored on the storage medium 14. The computer program 16 has the computer-generated steps of processing evidence from a scene where a crime has occurred that contains DNA from a specific person to produce DNA data. There is the step of interpreting the data to infer a probabilistic evidence genotype. There is the step of deriving from the evidence genotype a first score distribution that describes the frequency of scores from reference genotypes of people who did not contribute their DNA to the evidence. There is the step of deriving a second score distribution from the evidence genotype that describes the frequency of scores from reference genotypes that are probabilistically similar to the genotype of the person who contributed their DNA to the evidence. There is the step of constructing a curve from pairs of error rates at a plurality of scores, where each error rate pair comes from the first and second distributions evaluated at the score. There is the step of finding an area under the curve. There is the step of using the area as a measure of the score's ability to accurately discriminate between people who did or did not leave their DNA in the evidence. There is the step of organizing the genotype's score distribution pair, curve, and area into a discrimination package. There is the step of comparing the evidence genotype with the person's genotype to produce an inclusionary score; wherein the score is presented, together with a component of the package, in a legal proceeding causing conviction of the person of the crime, based on the score presentation, and incarcerating the person. This can also be used to acquit a person.

DNA is a powerful scientific tool for criminal justice, in principle able to determine whether or not someone has visited a crime scene. Thirty years ago, most usable biological evidence was simple, containing abundant DNA from one person. The laboratory data was unambiguous, producing a definite genotype without uncertainty. Following comparison with a reference genotype, the binary match result would either include or exclude a person of interest.

DNA matches require frequency statistics because some genotypes are common while others are rare (1). The original DNA match statistic was random match probability (RMP), a false positive error rate (ER) for chance misidentification. The RMP is a probability of misleading evidence (PME) (2) that someone who isn't the actual DNA contributor adventitiously matches the evidence.

The RMP reciprocal is a likelihood ratio (LR) that quantifies the evidential change in probability (3) of the hypothesis (H) that someone contributed their DNA to biological evidence. Before testing DNA, the prior probability is the RMP error rate. After seeing the DNA data and establishing a match, the posterior probability is one. Hence, for simple DNA evidence, the LR=1/RMP. Scientists use log₁₀(LR) as the weight of evidence (4).

But most evidence is not simple. Rather, DNA is usually a mixture of two or more people. Many items contain little DNA. On these complex DNA items, classical simple DNA interpretation fails. The data exhibit random variation and artifacts arising from the polymerase chain reaction (PCR) that amplifies DNA to detectable levels. For ease of interpretation, forensic scientists imposed artificial constraints, selecting and reducing DNA data to fit simple rules (5, 6). Such binary data reduction discards considerable identification information (7-9).

Forensic thresholds simplify DNA data. An analytical threshold discards all data peaks below a preset height, with many interpretation methods ignoring peak height as well (10). A stochastic threshold discards genetic locus test results when peak heights do not reach a (different and higher) level (11). A reporting threshold discards the entire DNA analysis if the LR match statistic is too small. As a result, simplistic interpretation of complex DNA data prevents informative evidence from ever reaching law enforcement or court (8). Limited data analysis strips away the truth-finding benefits of DNA evidence from both defendant (12) and state (13).

Unlike black and white thresholds, non-binary probability can render genotype possibilities in shades of gray, preserving DNA identification information. Random variables that capture PCR variation are used in hierarchical Bayesian models (14) to deliver accurate posterior probability for uncertain genotypes (15). Objective computer inference unmixes DNA mixture data into separated contributor genotypes. Posterior genotype probabilities are subsequently compared with references to yield LR values that numerically summarize evidential match strength (16).

Removing artificial interpretation limits lets in all the DNA data and uses non-binary likelihood functions (17). With more sophisticated data examination, scientists can report all their LR findings. Setting (by forensic convention) a zero log(LR) boundary value, a negative exclusionary log(LR) value classifies a comparison reference as a non-contributor, while a positive inclusionary score indicates a contributor.

Comparing an uncertain evidence genotype with every possible reference, and collating the log(LR) values into a histogram, would form a complete Noncontributor distribution. This bar chart describes the range and shape of exclusionary LR values to expect from everyone in the world who didn't contribute their DNA to the evidence. Conversely, comparing the uncertain genotype with all references likely to have produced that genotype gives the complete Contributor distribution of log(LR) values for probable DNA contributors.

These distributions characterize the real-world match statistic consequences of the DNA evidence. Sampled versions have been constructed by tedious time-consuming LR calculations on very small reference subsets (18). But, given their forensic importance and statistical power, a faster and more complete construction was needed. The previous invention provided a novel approach to constructing complete and exact LR distributions, as published (19). That direct construction used rapid convolution of probability functions, considering all possible 10²⁴ genotypes without making any reference comparisons or LR calculations.

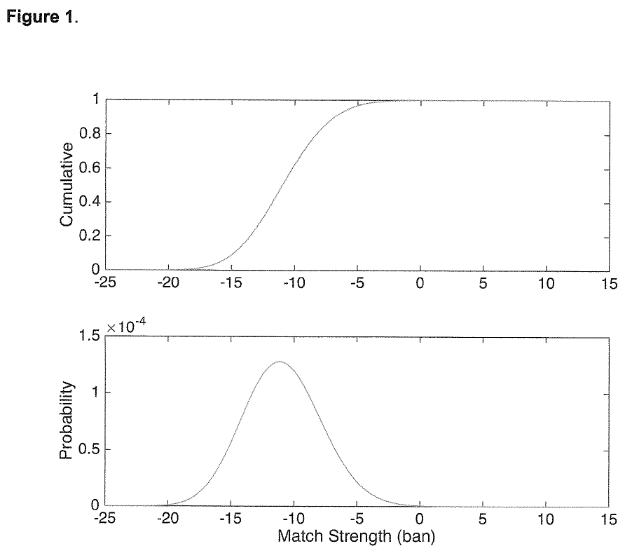

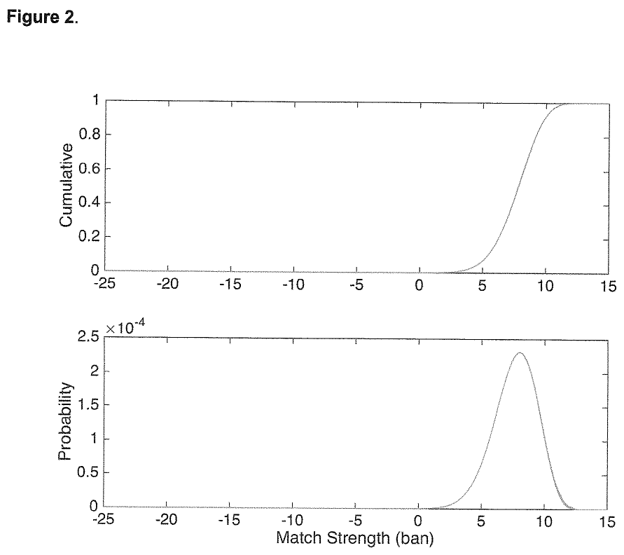

A genotype's exact Noncontributor log(LR) probability distribution is shown in FIG. 1 as both cumulative and density functions. FIG. 2 shows the corresponding Contributor distributions. The distribution average is the Kullback-Leibler (KL) divergence (20) that gives the expected log(LR) nonmatch (Noncontributor) or match (Contributor) information. Larger KL values correspond to more informative genotypes.

The previous invention showed how to merge a set of genotypes to yield a composite distribution, as published (19). These composites are useful in validation studies to summarize the DNA information that a laboratory produces. Looking up a distribution value immediately provides a PME error rate for a reported LR value. The ER accords frequency meaning to the LR, indicating how often the LR value might be misleading (19). Fora Contributor-classified inclusionary LR, a Noncontributor right tail probability evaluated at the log(LR) gives the ER. And, symmetrically, for an exclusionary Noncontributor LR the left Contributor tail is used.

An error rate helps assess confidence in a reported LR. For a single evidence genotype, the PME ER quantifies how often a LR might misclassify an alternative hypothesis. Scientists use ERs for process validation and statistical assessment of empirical testing results (21). Error rate is one of four Daubert prongs a U.S. judge considers when deciding the reliability of scientific evidence (22). An LR distribution collates these error rates at all log(LR) values into a single easily-computed function (19).

Signal detection theory determines the discrimination power and accuracy of a score-based classification method. The receiver operating characteristic (ROC) method is used to assess and compare the discrimination ability of different data models. ROC is well-established and pervasive in many fields, including medicine (23), psychology (24) and artificial intelligence (25). Forensic scientists use ROC to assess, compare and improve feature-comparison techniques (26), and validate DNA interpretation software on large genotype sets (27).

With forensic DNA the score is log(LR) and the two output classifications are Noncontributor and Contributor. Forensic interpretation software couples a probability model with DNA input data to produce an uncertain genotype. The genotype's prior and posterior probabilities (or likelihood) define unique Noncontributor and Contributor LR distributions (19).

Continuing further, the instant invention combines the genotype's two LR distributions into one ROC curve. It then finds the genotype's classification accuracy as the area under the curve (AUC). Unlike previous methods, this new dual distribution analysis derives a statistical framework from and for a single genotype. The framework measures the genotype's discrimination ability and provides LR error rates.

The instant invention centers on the reliability and reporting of LRs for uncertain genotypes. The previous invention described an efficient procedure for extracting a genotype's LR distributions at high resolution. That novel computation proceeded directly from genotype probabilities, without making any LR reference comparisons. Here, the specification presents diverse casework situations, applying the distribution concepts to forensic practice in both inclusionary and exclusionary settings. It also enables the combination of LR distributions in a novel and nonobvious way to measure and compare genotype discrimination accuracy.

### 2. Methods

This section summarizes methods from the previous invention, as published (19), which contains additional background and technical details. The section then presents a new method that measures a genotype's signal detectability directly from its LR distributions. It derives some useful properties of a LR-based score in ROC analysis.

### 2.1. Bayesian Genotyping

Genotyping data is produced by short tandem repeat (STR) (28) testing of multiple genetic loci by polymerase chain reaction (PCR) (29) amplification of template DNA molecules. PCR is a random branching process (30) that introduces inherent uncertainty into molecular copying (31). Quantifying electrophoretogram (EPG) signals from length-separated STR amplicons yields base pair size and relative fluorescence unit (RFU) amount data for locus alleles and other detected peaks.

Simple EPG interpretation attempts to classify STR data peaks into allele signals, background noise, and PCR artifacts (32). More sophisticated methods instead explain the STR locus pattern data under many different assumptions, without requiring classification.

Probability update begins with a prior genotype probability p(ω), for every genotype ω in the set Ω of possible genotypes. A likelihood function λ(ω) assesses how well varying genotypes ω, along with other variables, explain all the given locus STR data in the DNA evidence. Bayes Theorem (33) updates the prior p(ω) to a posterior genotype probability q(ω), for all ω in Ω, as the likelihood-prior product λ(ω)·p(ω), normalized to sum to one.

Genotype inference can be done in a hierarchical Bayesian model (14) that accounts for genotypes, and other relevant explanatory variables and their variance (15). Given STR input data, Markov chain Monte Carlo (MCMC) (34, 35) sampling from the variables' joint posterior probability distribution can solve for the variables, including q(ω). The practical consequence is that highly complex DNA mixtures containing many contributors, or having small amounts of DNA, can be computationally resolved (up to probability) into accurate component genotypes q (36) suitable for comparison.

Suppose there are K contributors to a DNA mixture, there is some STR kit contains L genetic loci, and there is a mixture M comprised of physical DNA sequences contributed by the K people. A laboratory generates STR data from the physical DNA mixture M. Genotype inference unmixes the physical mixture's data into the DNA sequences of each person, up to probability. For each of the K contributors, at each of the L genetic loci, the posterior genotype distribution q(ω) assigns a probability to each feasible genotype value, where an allele pair value is comprised of one or two physical DNA sequences. Therefore, a mixture contributor's genotype variable at locus corresponds to pairs of physical DNA molecules.

### 2.2. Likelihood Ratio

Genotype comparison between an evidence contributor and a reference is numerically summarized using a likelihood ratio (LR). The LR is the prior-to-posterior change in probability q(ω)/p(ω) for a genotype reference ω in Ω (37). The LR logarithm provides a score s(ω)=log₁₀(q(ω)/p(ω)) for the match strength between an evidence contributor genotype (with posterior probability q) and a person having genotype ω.

This standard log(LR) measure of information (4) quantifies how DNA evidence changes genotype probability. Forensic scientists conventionally impose a log(LR) inclusion/exclusion boundary of zero. A positive score (LR>1) can then be reported with the inclusionary language “a match between the evidence and reference is [LR number] times more probable than a coincidental match.” A negative score (LR<1) for exclusionary results has similar language, replacing “more” with “less”.

The LR relation to DNA amount is linear. Empirical studies show that log(LR) magnitude is proportional to log([DNA]) (15, 36). Here [DNA] is defined as the amount of DNA in a contributor to the biological evidence, where the LR is obtained by comparing that evidence contributor's genotype to a reference genotype. More DNA generally yields a larger inclusionary s(ω) to the true contributor ω, and a larger exclusionary score magnitude |s| to someone else.

Comparing a posterior genotype variable for a DNA mixture contributor to a reference genotype (relative to a population) assesses to what extent the physical biological evidence contains the physical DNA molecules contained in the reference person's body. The LR statistic measures the extent of physical connection between the evidence DNA molecules and the person's DNA molecules.

### 2.3. LR Distributions

Genotype distributions inherently arise from a Bayesian-inferred genotype. Imposing prior probability p on the genotype space Ω defines the Noncontributor random variable (RV) X:ω→s(ω) of prior-weighted log(LR) scores (19). The X cumulative distribution function (CDF) F_{X}(x)=Pr{X<x}, and its probability mass function (PMF) differences f_{X}(x), are shown in FIG. 1. Similarly, posterior probability q induces the Contributor RV Y:ω→s(ω) for posterior-weighted scores, with Y's CDF F_{Y}(x)=Pr{Y<x} and PMF f_{Y}(x) shown in FIG. 2.

The previous invention showed how to rapidly and accurately construct these real-valued probability distributions for X and Y by convolution as sums of independent locus variables (19). From Lyapunov's Central Limit Theorem (CLT), the distribution limit of these RV sums are normal (37). The X and Y PMF density curves cross at zero (Section 4.7, Proposition 2).

The average X and Y values give the Kullback-Leibler (KL) divergence (20), or relative entropy information difference, between prior and posterior probability. Specifically, −KL_{X}=−Eₚ[log(p/q)] for Noncontributor X, while KL_{Y}=E_{q}[log(q/p)] for Contributor Y. The credible interval of X or Y is defined by the distribution's highest posterior density (HPD) at a given probability level (38), providing a real-valued interval of LR domain support.

A composite distribution (39) aggregates N genotype distributions into a single FN distribution by CDF or PMF averaging (19). A validation study can analyze a composite distribution formed from a set of similar evidence genotypes to understand the DNA information produced by a laboratory process.

### 2.4. Error Rates

The probability of misleading evidence (PME) is the chance of observing strong LR evidence supporting one hypothesis over another when the hypothesis is false (2). The PME provides an error rate (ER) for an observed comparison LR, assessing the score in a frequency context. A definite genotype's PME is the familiar RMP error rate.

Generalizing to uncertain genotypes, the inclusionary PME_{Y} becomes the prior-weighted size of the smallest genotype set containing the matching reference ω. For a positive x=s(ω)>0 genotype comparison, the PME_{Y} is Pr{X≥x}. A negative x<0 score has an exclusionary posterior-weighted PME_{X} of Pr{Y<x}.

The likelihood ratio bounds the error rate (2). A definite genotype has an inclusionary LR error rate of ER_{Y}=RMP=1/LR. For uncertain genotypes, this strict equality relaxes to the Markov-Turing inequality ER_{Y}=PME_{Y}≤1/LR. The upper bound on error rate follows from Markov's inequality (30), together with Turing's observation that the expected value of an LR's alternative hypothesis is one, since Eₚ[q/p]=Σq=1 (4). The 1/LR bound has empirical support (40).

For an exclusionary LR, the relation ER_{X}=PME_{X}≤LR holds. The proof inverts LR q/p to p/q and flips x to 1/x (41). Combining this LR upper bound on error rate, with the proportionality between LR and DNA amount |log(LR)|≈log([DNA]), yields a rough error rate upper bound by DNA amount ER≤1/[DNA]. Thus, LR error rate generally decreases with increasing DNA contributor amount, for both inclusionary and exclusionary match strength scores.

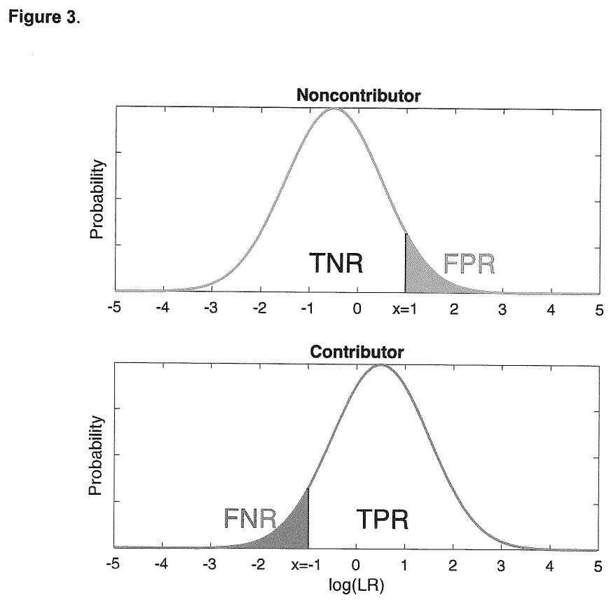

The distribution tail probability provides genotype comparison LR error rates, whether directly read as a CDF value, or calculated as a PMF area. For an inclusionary score x>0, the false positive rate (FPR) Contributor ER_{Y}(x) at x is the Noncontributor right tail probability Pr{X≥x}=1−F_{X}(x), shown in FIG. 3 (red). One can state ER_{Y} as “for a match strength of [LR], only 1 in [1/ER_{Y}] people would match as strongly.” The [1/a] reciprocal convention rewords a fraction 0<a<1 as a counting number. The true negative rate (TNR) at x is 1−FPR, or Pr{X<x}=F_{X}(x).

For an exclusionary score x<0, the false negative rate (FNR) Noncontributor ER_{X}(x) at x is the Contributor left tail probability Pr{Y<x}=F_{Y}(x), shown in FIG. 3 (blue). ER_{X} can be verbally expressed as “for an exclusionary statistic of one over [1/LR], only 1 in [1/ER_{X}] people would be excluded as strongly.” The true positive rate (TPR) at x is 1−FNR, i.e., the Contributor right tail probability Pr{Y≥x}.

### 2.5. Signal Detection Theory

Receiver operating characteristic (ROC) analysis measures a method's discriminating ability, independently of decision threshold and condition prevalence (42). With DNA matching, the decision parameter is the log(LR) match statistic score. The two real-world conditions in this context are whether or not someone contributed their DNA to biological evidence. These concepts are connected by the Noncontributor X and Contributor Y LR genotype distributions presented in the error rate definitions and equivalences of Table 1.

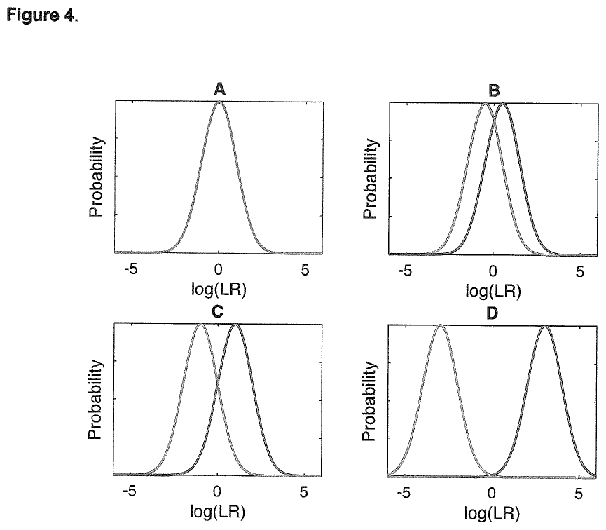

The paired Noncontributor X and Contributor Y score distributions shown in FIG. 4 have varying degrees of curve overlap. Complete overlap (A) means there is no discrimination capability based on the x-axis score. More separated distribution pairs (C) can better discriminate scores than less separated pairs (B). Complete curve separation provides perfect score-based condition discrimination (D).

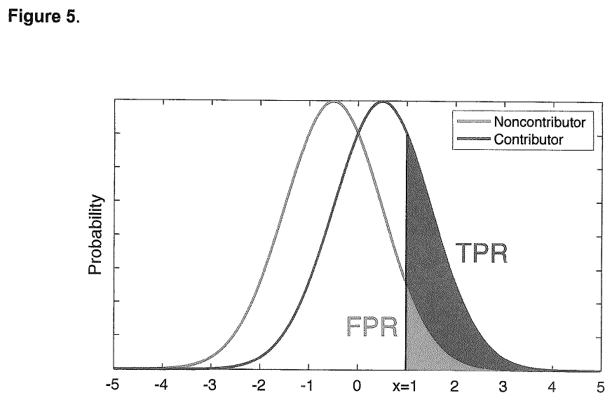

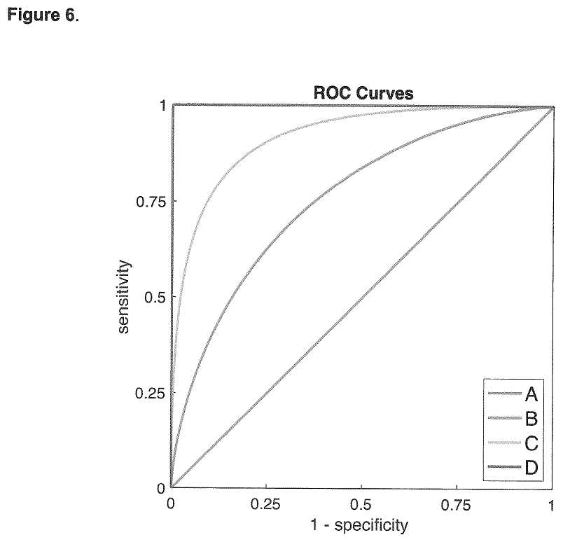

An ROC curve couples specificity X-derived and sensitivity Y-derived error rates as paired (FPR(x), TPR(x)) coordinate points, depicted in FIG. 5. These two-dimensional points are parameterized by a varying one-dimensional x=log(LR) score. The four ROC curves of FIG. 6 correspond to the identically labeled Noncontributor/Contributor distribution pairs of varying discriminating ability shown in FIG. 4.

The FIG. 6 straight ROC line A indicates total distribution overlap, with equal false and true positive rates at all score values, providing no discrimination ability. As the ROC curves B and C bend away from uninformative line A toward the error-free top-left corner point (0,1), discriminating capability increases. ROC curve D shows a perfect discriminator, with zero FPR along the left y-axis and a TPR of one along the top edge, regardless of score.

ROC analysis quantifies a model's discrimination ability as the Area Under the Curve (AUC). An uninformative model's 45° ROC curve area fills only half the square (lower right triangle), for minimal AUC=0.5 (A). As discrimination capability increases, so too does the AUC. B's ROC curve has AUC=0.7602, while C's more accurate curve has AUC=0.9831. A perfect discriminator has maximal AUC=1, since its ROC curve area (D) fills the entire FPR×TPR unit square.

The AUC accuracy measure is Pr{X<Y}, the probability that a randomly selected pair of scores (sₓ, s_{y}) drawn from each of the Noncontributor and Contributor distributions will be ordered correctly, i.e., sₓ<s_{y}. For example, an AUC of 0.9 implies that 10% of the time a pair of scores would inaccurately confuse the true classifications. The proof involves a change of variables in the AUC integral (43).

The ROC AUC accuracy statistic tells criminal justice decision makers to what extent they can rely on a reported LR match statistic. An AUC near 1 says that an inferred genotype can reliably determine whether or not a suspect's DNA molecules are contained in biological evidence. An AUC far from 1 says that the genotype cannot as reliably distinguish a DNA inclusion or exclusion. AUC confidence in an inculpatory LR match statistic can lead police, prosecutors, judges, and juries to confidently arrest, try, convict, and incarcerate a guilty criminal. High confidence in an exculpatory LR value can lead them to release, acquit, or exonerate an innocent person. Low AUC confidence can help them ignore less reliable DNA evidence to avoid a miscarriage of justice. The ROC AUC is directly connected to actions people take that decide the criminal justice fate of a human suspect or defendant.

Different forensic scientists may have different cost/benefit risk preferences. One crime laboratory might be equally comfortable with a false negative as with a false positive. Another lab may find not missing a positive match (to not free a criminal) ten times more important than mistakenly identifying an innocent person (and arresting them unnecessarily). ROC analysis can set a decision score threshold to accommodate such cost preferences. Its binary threshold can also account for prior probability. A cost-adjusted binary score decision threshold can be determined using ROC curve tangent lines or algebraically. These customizations are built into many statistical programming languages. For example, MATLAB's perfcurve ROC function accepts both ‘Prior’ and ‘Cost’ score customization inputs.

### 2.6. Statistical Framework

A Bayesian-inferred genotype implicitly defines a statistical framework for reference comparisons. The genotype's prior p and posterior q probability distributions mathematically imply a pair of easily computed Noncontributor X and Contributor Y LR score distributions (19). ROC analysis gives the AUC discrimination ability of this LR distribution pair. These genotype-derived LR distributions and their accuracy are known before any comparison is made to a reference genotype ω.

Each genotype comparison produces a log(LR) score x=s(ω) that numerically quantifies the match strength between DNA evidence and reference. Error rates for this LR can be derived from the genotype's Noncontributor-Contributor statistical framework. Using the conventional forensic LR=1 boundary, the FPR(x) is 1−Fₓ(x) for inclusionary x>0, and the FNR(x) is F_{y}(x) for exclusionary x<0. These statistical error rates are automatically derived from the evidence genotype itself, independently of laboratory validation or reference comparisons.

### 2.7. ROC Likelihood Ratio

The ROC curve has a likelihood ratio LR_{ROC}(x) defined by the f_{Y}(x)/f_{X}(x) tangent line slope at graph point ROC(x). One can show that this LR_{ROC}(x) equals LRₓ, the Bayesian LR. That is, an LR-based categorization score replicates the LR value in its ROC curve. It can also be shown that the X and Y PMF curves cross at x=0, and that this point is the optimal ROC threshold. ROC is invariant to monotone transforms (43), so the propositions here also apply to the LR score and all its monotone score transformations, not just the logarithm. The discrete proof approximations sharpen with increasing bin resolution and genotype sampling (19); continuous versions use ROC derivatives (44).

Definitions. For any genotype ω∈Q, LR(ω)=q(ω)/p(ω) (19, § 2.3). The match strength categorization score s(ω) is log[LR(ω)]. The real-valued distribution functions F_{X} and F_{Y} have a score domain where x=s(ω), and LRₓ=LRₓ(ω)=10^{x}, for ω∈Ω.

Let the genotype pre-image Sₓ be the iso-LR subset {ω∈Ω|s(ω)=x}. Fix a bin resolution ε (19, § 2.4). At that resolution, for random variables X and Y, there are the approximate probability densities

$$\begin{matrix}{f}_{X} ( x ) = {μ}_{p} [ {S}_{x} ] = {\sum }_{ω ∈ {S}_{x}} p ( ω ) , and \\ {f}_{Y} ( x ) = {μ}_{q} [ {S}_{x} ] = {\sum }_{ω ∈ {S}_{x}} q ( ω ) = {\sum }_{ω ∈ {S}_{x}} {LR}_{x} \cdot p ( ω )\end{matrix}$$

since LRₓ is constant on Sₓ.

Proposition 1. The LR_{ROC}(x) of the ROC curve at x equals the Bayesian LRₓ value.

$$\underline{Proof} . {LR}_{ROC} ( x ) = \frac{{f}_{Y} ( x )}{{f}_{X} ( x )} = \frac{{\sum }_{{ω ∈}_{} {S}_{x}} q ( ω )}{{\sum }_{ω ∈ {S}_{x}} p ( ω )} = \frac{{\sum }_{{ω ∈}_{} {S}_{x}} {LR}_{x} \cdot p ( ω )}{{\sum }_{ω ∈ {S}_{x}} p ( ω )} = \frac{{LR}_{x} \cdot {\sum }_{ω ∈ {S}_{x}} p ( ω )}{{\sum }_{ω ∈ {S}_{x}} p ( ω )} = {LR}_{x}$$

Proposition 2. The X and Y PMF density curves cross at x=0 (i.e., LRₓ=1).

Proof. At a PMF curve crossing, f_{X}(x)=f_{Y}(x) implies f_{Y}(x)/f_{X}(x)=1. But then LRₓ=LR_{ROC}(x)=f_{Y}(x)/f_{X}(x)=1. And so, x=log₁₀[LRₓ]=log₁₀(1)=0. Therefore f_{X}(x)=f_{Y}(x) exactly when x=0.

Proposition 3. The optimal ROC threshold for distinguishing Noncontributor and Contributor categories is x=0 (i.e., LRₓ=1)

Proof The task is to maximize the positive rate separation PRS(x) between the survival functions TPR and FPR (45). In terms of cumulative distributions Fₓ and F_{y}, PRS=TPR−FPR=(1−F_{y})−(1−Fₓ)=Fₓ−F_{y}.

Differentiating yields a probability density function difference

$$dPRS / dx = {f}_{x} - {f}_{y}$$

Setting the derivative to zero to maximize PRS gives

$${f}_{x} - {f}_{y} = 0 , or {f}_{x} = {f}_{y}$$

From Proposition 2, the functions are equal when x=0. Therefore, the optimal ROC threshold for Contributor categorization is x=0.

This optimal threshold derivation assumes equal weighting of sensitivity and specificity. There are more general objective functions that consider the relative costs of classifying true positives and negatives (46). A more complete decision-making formulation can incorporate overhead cost and each of the four true/false and positive/negative decision costs (42). With unequal category weighting (47), the optimal s(ω)) score cutoff would generally deviate from LRₓ=1.

### 3. Materials

### 3.1. Software Programs

The fully Bayesian TrueAllele® Casework system (Cybergenetics, Pittsburgh, PA) separates STR mixture data to produce a genotype for each DNA contributor (15). A TrueAllele server inputs DNA data, transforming genotype probability from prior to posterior. The VUIer™ client software's Report module plots these probabilities and calculates LR match statistics.

The Distribution View in Report constructs, displays and exports Noncontributor and Contributor distributions, along with their summary statistics (19). Distribution rendered all the genotype distribution figures in this paper. TrueAllele versions 3.25.5840.1 or older (server) and VUIer version 3.3.8663.1R20b (client) were used in this work.

TrueAllele is written in the MATLAB programming language (The Mathworks, Natick, MA). MATLAB Version R2022a was used to perform and display the ROC analysis. Probabilistic genotyping (PG) STRmix™ software (ESR, New Zealand) version 2.8.0 was run in the Sandoval case. The BAYES analysis (version 0.9) for diagnosing emergency room patient chest pain (Vaizian, Chicago, IL) was conducted using Vaizian's free online webapp (48).

### 3.2. Low LR Cases

Previously run TrueAllele cases were collected that had a relatively low LR magnitude of |log(LR)|≤6. 95 inclusionary genotypes were identified in this set with positive log(LR) scores. These genotypes came from 52 consecutive cases in 15 US states and 1 territory, comprising 91 evidence items analyzed in 24 crime laboratories using diverse STR kits and DNA sequencers (Table 2, inclusionary). The items were submitted to Cybergenetics for TrueAllele computer interpretation by 28 police and 17 prosecution agencies (including the FBI) between January 2022 and November 2024. The genotypes had 1 to 6 contributors, averaging 3.41 contributors (Table 3, inclusionary).

Similarly, 85 exclusionary genotypes were identified in the set that had low negative log(LR) match statistics. The genotypes came from 51 consecutive innocence or criminal defense cases. The 77 evidence items had been analyzed by 30 crime laboratories using various STR kits and DNA sequencers (Table 2, exclusionary). The items were submitted to Cybergenetics for TrueAllele screening by 7 innocence or post-conviction integrity units, and 44 defense lawyers or agencies between April 2019 and November 2024. The genotypes contained 2 to 6 contributors, averaging 3.19 contributors (Table 3, exclusionary).

### 3.3. Highlighted Cases

The specification highlights the role of LR distributions in five criminal cases. Cybergenetics ran TrueAllele on the case DNA evidence items. United States v. Curtis Johnson was a federal prosecution case with low-level DNA mixture evidence and a small inclusionary LR value. A Daubert hearing established TrueAllele admissibility before the jury heard DNA testimony. State of Georgia v. Kerry Robinson was a post-conviction case involving a small exclusionary LR from a DNA mixture that led to the exoneration of an innocent man.

In United States v. Alejandro Sandoval the defense utilized two PG software solutions, TrueAllele and STRmix. Both programs produced exclusionary LRs, but they were of different magnitudes, stimulating discussion in the forensic science community (49). In United States v. Ravel Mills a US attorney made a novel unfounded argument against a federal defender's TrueAllele report. Cybergenetics' scientific response to this unsuccessful Daubert challenge used LR distributions. In the United Kingdom, Regina v. Stuart Burton showed how LR error rates could help resolve multiple hits to a DNA database.

### 3.4. DNA Data Collections

Cybergenetics processed electronic data files, not biological material. The company received the electronic STR data for the cases in this study as DNA sequencer output files, in either .fsa or .hid file format. Data sets were deposited (with permission) on Mendeley Data (50) for US v. Sandoval (51), and also (52) for a DNA mixture validation study (15).

### 4. Results

The first four results sections center on LR distributions and ROC analysis for inclusionary DNA evidence (LR>1) in casework and validation applications. The next four sections reprise the analysis, but instead for exclusionary casework evidence (LR<1) and applications. The last two sections show how LR distributions and error rates allow scientists to move beyond arbitrary genotyping limitations imposed by binary methods.

4.1. United States v. Johnson Conviction

On Dec. 18, 2013, an armored truck was robbed outside a New Orleans bank. The guard was killed in a shootout. A bandana left at the crime scene contained a 70 pg low-level DNA mixture. Initial comparison of the three-person mixture data with suspect Curtis Johnson was inconclusive. Five years later, TrueAllele computer reinterpretation of the same mixture data derived three genotypes. The software connected a 27% contributor to Johnson. As reported in United States v. Johnson, “a match between the bandana and Johnson was 200 times more probable than a coincidental match,” placing Johnson at the scene with a LR of 200.

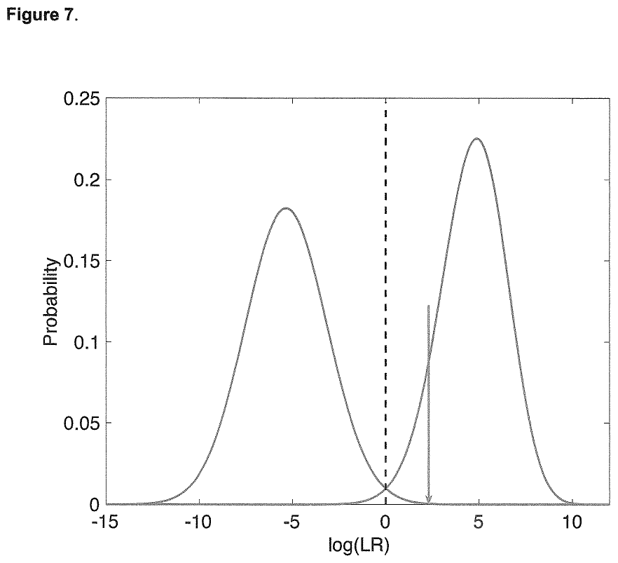

The evidence genotype's Noncontributor (left) and Contributor (right) LR distributions are shown in FIG. 7 (blue curves). Their respective KL values were −5.32 and 4.67 ban, with AUC=0.9997. The arrow (green) indicates Johnson's log₁₀(LR) of 2.30 ban. These distributions provide a frequency context for the defendant's LR, relative to all possible reference genotypes (19). The right tail area of the Noncontributor distribution gave an ER of 1 in 4,100, or log₁₀(ER)=−3.61. As reported, “for a match strength of 200, only 1 in 4.1 thousand people would match as strongly.”

The defendant challenged the evidence, claiming “the TrueAllele methodology has not yet been proven reliable or generally accepted, especially when analyzing very small amounts of DNA.” At the Daubert hearing a scientist presented evidence of TrueAllele reliability, including 42 validation studies, 22 of which examined small DNA amounts. The judge admitted TrueAllele as reliable evidence, allowing the jury to hear the DNA evidence and TrueAllele statistics. Johnson was convicted of murder and sentenced to 50 years in prison.

### 4.2. Validating Inclusionary LR Results

Cybergenetics the sensitivity and specificity of low inclusionary match statistics. TrueAllele developed 95 genotypes from sequential DNA items submitted to the company by law enforcement that showed LRs between one and a million. The items had 1 to 6 contributors, averaging 3.41 (s=1.32). Contributor mixture weights were from 0.01 to 1, with x̅=0.27 (s=0.23). LR logarithms ranged from 1.12 to 5.99 ban, having x̅=3.26 (s=1.39).

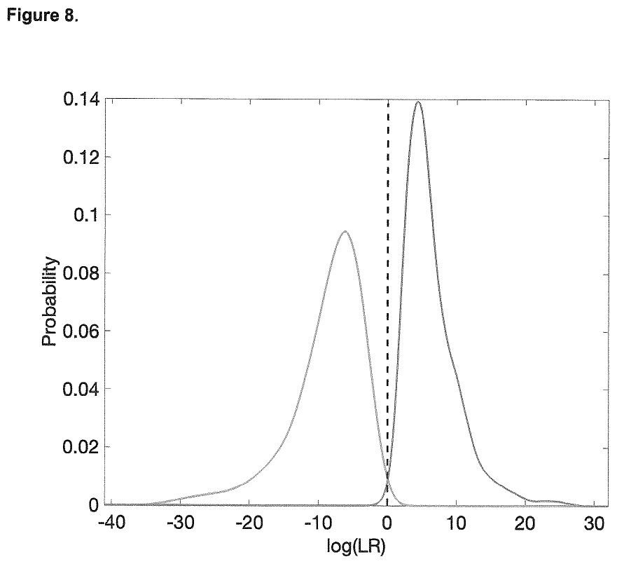

The composite evidence genotype's Noncontributor (left red curve) and Contributor (right magenta) LR distributions are shown in FIG. 8 (19). These composite distributions are quite similar to the case distributions of FIG. 7. Over the validation genotype set, the composite contributor KL is 6.32 ban, for an average inclusionary LR of over a million. The informative Noncontributor −KL of −8.81 ban helps establish low error rates. There is little distribution overlap, as sensitivity and specificity both exceed 99%, with AUC=0.9998.

These composite distributions can be used to assess the bandana LR of 200 in US v. Johnson, relative to a validation set of similar low LR match statistics. At log(LR)=2.30, the Noncontributor distribution had a right tail probability of 1 in 4.31 thousand, or log(ER)=−3.63. The validation error rate is very close to the case log(ER) of −3.61.

### 4.3. Inclusionary LR Error Rates

An inclusionary LR's error rate ER is necessarily bounded above by its reciprocal 1/LR. Moreover, empirical studies show that the logarithms of LR and contributor DNA amount are proportional. Therefore, for a computationally unmixed genotype, a contributor's ER varies in inverse proportion to its DNA amount. More contributor DNA reduces ER, while less DNA increases ER.

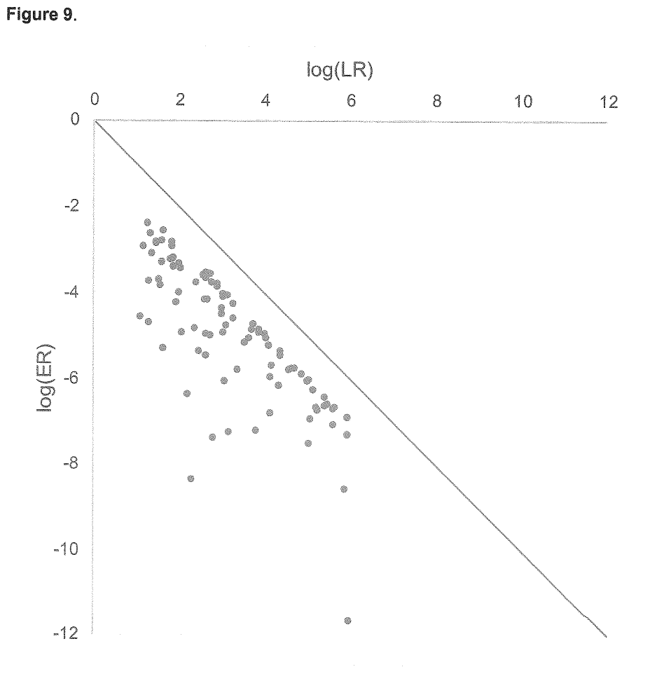

The exact ER of a suspect's inclusionary LR can be quantified using the evidence genotype's Noncontributor score distribution (19). The FIG. 9 log-log scatterplot shows ER as a function of LR for the 95 low inclusionary LR values in the preceding section's validation study. Clearly, the ER≤1/LR upper bound holds. Many exactly computed ER values are orders of magnitude below the guaranteed Markov-Turing 1/LR upper bound, which can instill even greater confidence in the LR result.

The figure shows a gap between the upper edge of the scatterplot points, and the log(ER)=−log(LR) boundary line. The general Markov (and Chebyshev) inequalities are weaker than specific distribution bounds. The genotype log(LR) distribution X is a sum of independent locus distributions (19), so from the Lyapunov-CLT, X tends toward a normal distribution. A Lilliefors test (53) supports the normality of the Johnson Noncontributor distribution (p=0.50). Tighter bounds on a normal tail include a standard normal density φ(x) (54) and φ divided by x (55). The logarithm of φ has a linear term in x plus a constant term, reproducing the 1/LR Markov bound and accounting for the scatterplot gap, respectively. Others have reported similar log(φ) term separations (2).

### 4.4. Inclusionary Reliability at Low LR

Inclusionary LR error rates can be quantified using Noncontributor distribution tail probability (19). The score distribution can be derived from the particular evidence genotype used in a case LR comparison. In a validation study, a composite distribution can be constructed from a set of similar genotypes. Either approach can provide error rates that help establish the reliability of low LR match statistics, which are often seen with LT-DNA mixtures.

Table 4 shows PME error rates from Noncontributor distributions. The two parts show CDF probabilities formed from the example case genotype, and an ER column computed as 1−CDF. The left Case part is from the Johnson evidence genotype distribution, while the right Validation part is from the composite distribution of 95 inclusionary genotypes. Here, the case and validation distributions and ERs are similar, showing that the case genotype is representative of the low-template DNA validation genotype set.

The instant invention combines a genotype's Contributor and Noncontributor LR distributions to form an ROC curve whose AUC area quantifies the genotype's LR match accuracy. In Johnson, the ROC AUC match classification accuracy for the bandana genotype was 99.98%. Based on the high match confidence instilled by the ROC test, a jury would reasonably decide that the TrueAllele bandana LR of 400 implicating the defendant was reliable evidence, and convict him of the crime. Following the jury's ROC-based guilty verdict, the judge would sentence the convicted man to serve time in prison.

4.5. Georgia v. Robinson Exoneration

On Feb. 15, 1993, three teenagers broke into a 42-year-old Georgia woman's home and raped her at gunpoint. A suspect falsely accused 17-year-old Kerry Robinson (56). Limited binary review of STR data from a sexual assault kit couldn't fully untangle the three-person DNA mixture. On Feb. 26, 2002, based on faulty human mixture interpretation, Robinson was convicted of rape, and sentenced a month later to 20 years in prison.

On Sep. 5, 2018, Cybergenetics received the DNA data. TrueAllele derived three genotypes from the sperm-fraction mixture. The Bayesian computation connected the suspect and another defendant to the DNA mixture, but not Robinson. Reporting the smallest LR across four ethnic subpopulations, “a match between the vaginal and/or cervical swabs sperm fraction and Kerry Robinson was 103 times less probable than a coincidental match,” providing new exculpatory DNA evidence.

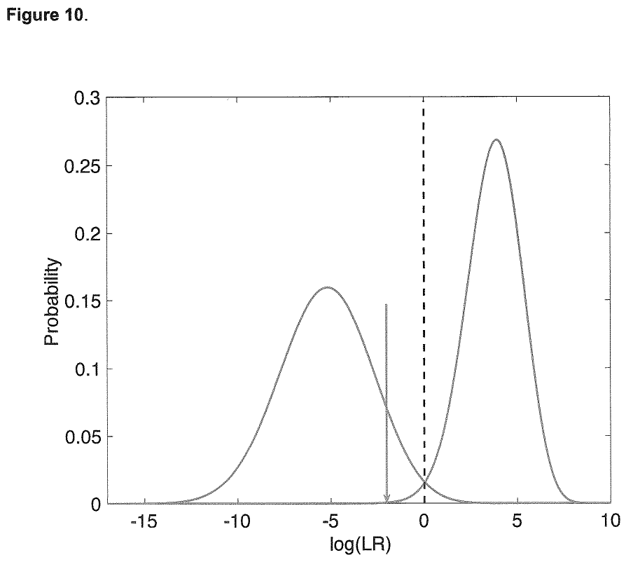

FIG. 10 (blue curves) shows the 10% contributor genotype's Noncontributor (left) and Contributor (right) LR distributions (19). The average log(LR) values were −5.27 and 3.73, respectively, with AUC=0.9993. The green arrow indicates Robinson's exclusionary log(LR) of −2.01 ban. The left tail area of the Contributor distribution provides an ER of 1 in 3,730, with log(ER)=−3.57. The error rate can be stated as “for an exclusionary statistic of one over 103, only 1 in 3,730 people would be excluded as strongly.”

On Jan. 8, 2020, relying on the new DNA evidence, a Colquitt County judge vacated Robinson's convictions and granted him a new trial. Robinson was released from prison that day, after serving almost 18 years for a crime he did not commit. The case was not retried.

The invention's ROC AUC match classification accuracy can be very high (99.93% in Robinson). This large ROC AUC can assist the court by instilling great confidence in a small exclusionary LR match statistic (in Robinson, 1 over 103). Confident in the DNA evidence, the judge would exonerate the convicted person, who would then be released from prison.

### 4.6. Validating Exclusionary LR Results

The low exclusionary match statistic sensitivity and specificity were studied. TrueAllele inferred 85 genotypes from sequential DNA items that defendants and innocence groups had submitted to Cybergenetics with LRs between one and a millionth. The items contained 2 to 6 contributors, averaging 3.19 (s=1.02). Contributor mixture weights were 0.01 to 0.50, with x̅=0.17 (s=0.14). LR logarithms ranged from −5.96 to −0.03 ban, having x̅=−3.54 (s=1.54).

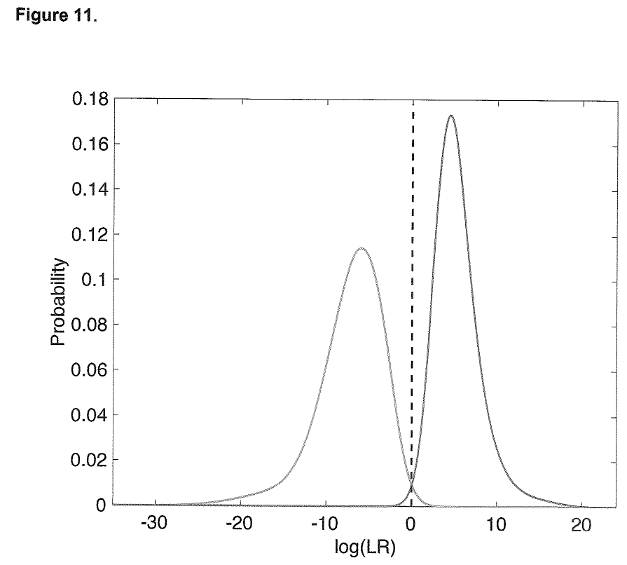

FIG. 11 shows the composite evidence genotypes' Noncontributor (left red curve) and Contributor (right magenta) LR distributions (19). Noncontributor −KL was −7.21 ban over the validation genotypes, for average exclusionary LR under a millionth. Contributor KL was 5.34 ban, providing low error rates. The distributions barely touch, with validation sensitivity and specificity both over 99%, and AUC=0.9997.

These composite distributions can provide a frequency context for the sperm fraction LR of 103 in the Robinson exoneration, relative to comparable low LR match statistics. At log(LR)=−2.01, the Contributor distribution left tail probability was 1 in 6.28 thousand, or log(ER)=−3.80. The validation error rate and the case log(ER) of −3.57 are similar.

### 4.7. Exclusionary LR Error Rates

An exclusionary LR's error rate is bounded above by the LR value. Larger DNA amounts generally yield smaller (i.e., more informative) exclusionary LR values less than one. Thus, the ER of a contributor genotype's exclusionary LR varies inversely with its DNA amount. More contributor DNA in the evidence lowers the ER of an LR comparison to someone else.

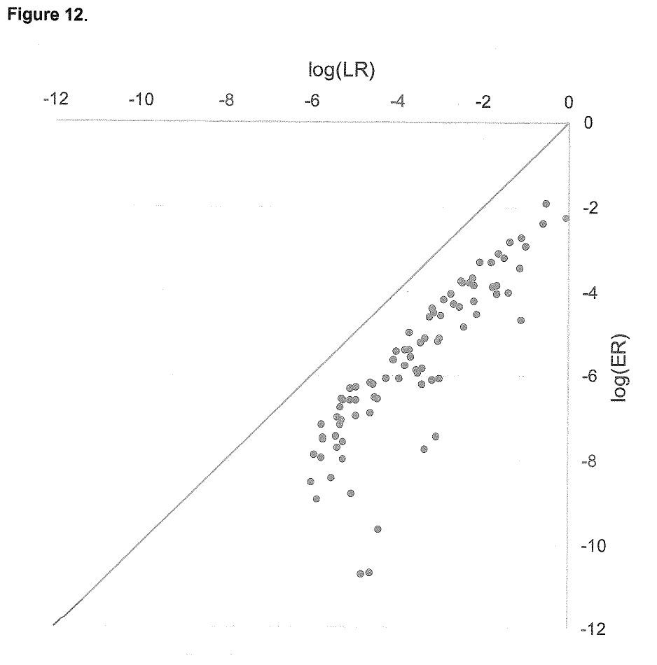

An evidence genotype's Contributor score distribution can be used to quantify an exclusionary LR's error rate (19). The scatterplot in FIG. 12 shows ER as a function of LR for the 85 low exclusionary LR values in the validation study of the preceding section. Comparing the third quadrant's doubly negative log values provided empirical support for the theoretical ER≤LR bound. When computed ER values are orders of magnitude less than the LR, they confer increased confidence in reported LR accuracy.

There is a gap between the scatterplot points and the exclusionary LR upper bound. The Contributor distribution's properties are symmetrical with Noncontributor (19). Thus, the above Noncontributor CLT argument applies to the Contributor sum of locus random variables, and its limiting normal distribution. The log(φ) left-tail bound has constant and linear (in LR) terms. The constant term explains the scatterplot gap, while the linear term restricts points to stay within their triangular boundaries.

### 4.8. Exclusionary Reliability at Low LR

The Contributor distribution tail probability enables error rate determination for exclusionary LR match statistics. Within a case, an exact LR distribution can be derived from an evidence genotype (19). In a validation study, aggregating groups of similar genotypes in a composite Contributor distribution can measure a laboratory's interpretation sensitivity under various conditions (19). Regardless, the resulting PME error rates provide a useful frequency context for low LR exclusionary match statistics, which often arise with LT-DNA and mixtures of many contributors.

Table 5 shows PME error rates from two Contributor distributions. For left tail probabilities, the PME error rate at an LR is just the CDF probability. The left part is from the Robinson case genotype, while the right is from the validation's 85 exclusionary genotype composite distribution. The case and validation score distributions and ERs are similar. Both follow the exclusionary ER≤LR Markov-Turing inequality.

### 4.9. Data Thresholds Discard DNA Evidence

Most forensic DNA interpretation relies on an analytic threshold (AT) that filters out data peaks having heights below a laboratory's preset AT value (10). This data reduction attempts to distinguish allelic data from baseline noise or PCR artifact. The TrueAllele technology doesn't need or use thresholds. Its Bayesian model accounts for noise and artifacts, letting the software consider all the data (15).

California v. Sandoval involved a DNA mixture swabbed from a package containing methamphetamine. TrueAllele inferred contributor genotypes, using 210 data peaks across 21 STR loci. The log(LR) to the defendant was −6.08 ban, relative to an African-American population. The error rate logarithm was −8.35 for this exclusionary match statistic.

The DNA lab that generated the STR data applied their STRmix PG program to the same evidence item (49). The lab's reported STRmix log(LR) match statistic to the defendant was a lower exclusionary −1.38 ban (AT=40 RFU, 24 input peaks), relative to a Southwest Hispanic population. At different data thresholds, STRmix log(LR) values ranged 7 orders-of-magnitude from −0.53 ban (AT=90, 11 peaks) to −7.48 ban (AT=20, 38 peaks).

Discarding DNA data by applying peak height thresholds can lose considerable identification information, particularly with low-level mixtures. More data reduction leads to greater information loss. LR distributions (19) can help explain how data input impacts LR information. The laboratory's choice of Southwest Hispanic population was used in the following LR comparisons.

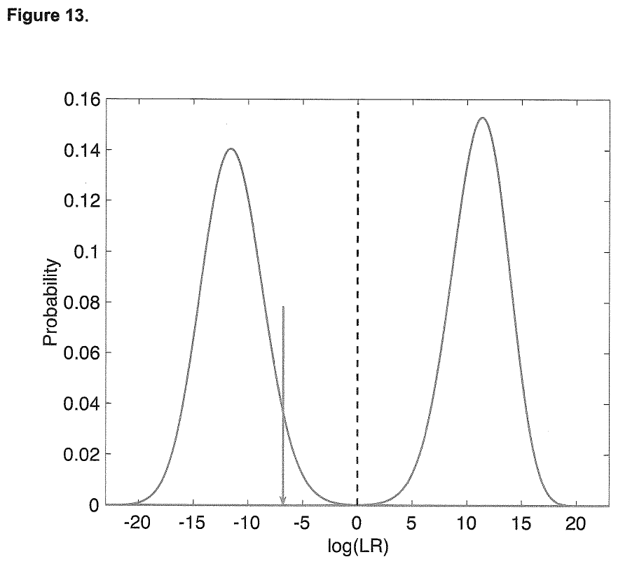

FIG. 13 shows TrueAllele's threshold-free LR distributions for the exclusionary genotype. The informative Noncontributor distribution (left) has 99.99% specificity, with an average log(LR) of −11.41. This −KL is consistent with the defendant's exclusionary log(LR)=−6.78 match statistic (green arrow), distancing the defendant from the evidence. The informative Contributor distribution (right) has 99.99% sensitivity, with KL=11.04. The FPR area to the left of the LR arrow gives a small error rate of log(ER)=−8.77.

The lab applied a 40 RFU peak-height threshold to form STRmix input data. The software then separated the minor component genotype, at each locus giving a likelihood function over allele pairs. These genotype likelihood results were loaded into TrueAllele to derive genotype probability, and therefrom compute LR distributions, match statistics, and error rates.

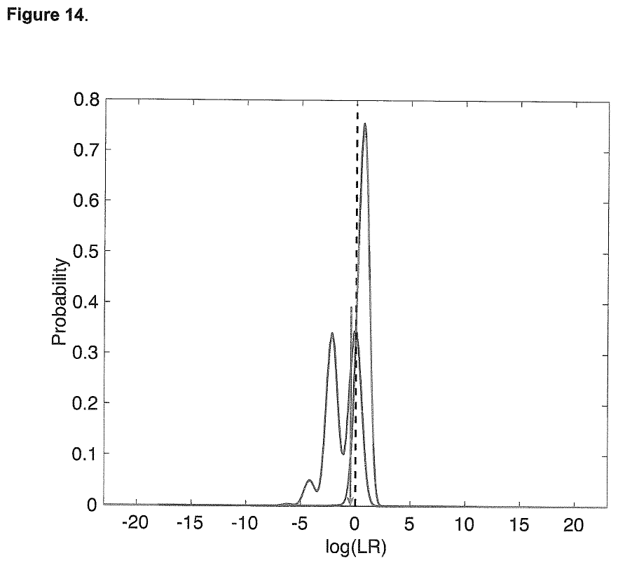

FIG. 14 shows the LR distributions for the STRmix genotype. The Noncontributor distribution (left) has 77.09% specificity and −KL=−1.26, near the recomputed log(LR)=−0.45 match statistic (green arrow). The STRmix Contributor distribution (right) has 88.17% sensitivity and KL=0.64. The distribution's left tail reveals an appreciable false negative LR error rate of ER_{X}=2.53%. For the exclusionary LR of 0.36, only 1 in 40 people would be excluded as strongly. The 99.9% HPD credible intervals of the Contributor and Noncontributor distributions overlap, with the LR falling within their intersection, suggesting a potentially uninformative result.

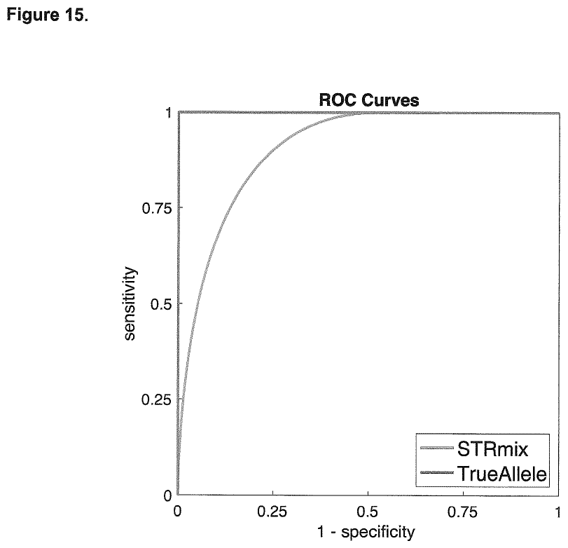

FIG. 15 shows a perfect classifier TrueAllele ROC curve (AUC=1, blue), with rectilinear edges overlying the x and y axes. A less discriminating STRmix curve (AUC=0.9077, red) is shifted inward. The genotype-derived LR distributions, ROC curves, and associated statistics quantify the adverse impact of data thresholds on DNA identification information in this case.

Table 6 shows how analytical thresholds can reduce DNA identification information. Threshold-free TrueAllele used 210 Sandoval peaks drawn from all 21 loci, producing well-separated Noncontributor (−KL=−11.42) and Contributor (+KL=11.03) distributions, with AUC=1. As the STRmix threshold was raised from 10 to 90 RFU, the number of input loci and peaks shrank. A narrowing [−KL, +KL] distribution interval gave diminishing AUCs. Typical threshold levels between 50 and 100 RFU produced small exclusionary log(LR) values between −1 and 0.

When multiple data analysis methods interpret complex DNA with varying LR results, the instant invention can help a court decide which method it should rely upon. In Sandoval, TrueAllele's inferred evidence genotype had a match classification ROC AUC accuracy of 100%, making its log(LR) of −6.08 ban that excluded the defendant extremely reliable. STRmix had a lower ROC AUC accuracy of only 91%, so its near-zero log(LR) of −0.45 (essentially inconclusive) was less reliable. Moreover, with higher RFU thresholds that reduce the data input to STRmix, the software's ROC AUC classification accuracy descended to a low of 65%—giving the wrong include/exclude answer a third of the time.

A judge can use the instant ROC AUC invention to choose the more reliable TrueAllele result (exclusionary LR, AUC=1) over the less reliable STRmix (inconclusive LR, lower AUC). The TrueAllele result statistically excluded the defendant, so the prosecutor dropped the more serious charge. Convicted of a lesser crime, the defendant received less time in prison.

### 4.10. LR Thresholds Produce Inconclusive Results

Many forensic DNA laboratories have a reporting threshold (RT) for LR match statistics. They declare smaller LR magnitudes under the LR threshold “inconclusive”, and do not report the results. A key motivation for their dismissing match information is to ensure a zero-error rate when reporting DNA results. A laboratory validation study will typically test genotyping software on dozens of DNA mixtures, comparing with hundreds of references. Some have compared with hundreds of thousands (57). By setting the RT higher than the largest observed false positive LR, the lab has some comfort they won't report a false DNA match.

However, the RT represents an under-sampling fallacy. A composite genotype CDF approaches 1 with increasing LR. Comparing the genotype set with a sufficient number of DNA references will eventually find a match whose LR exceeds the preset RT value, forcing the lab to raise their fixed RT bound. Accounting for discrimination ability using hundreds of “ground truth” mixture genotypes can help lower PG software RT to report more low LR evidence (58).

In the published Kern County validation study (59), 50 genotypes were derived from 10 5-person DNA mixtures. These 50 validation genotypes were compared with increasing numbers of R randomly generated reference samples, log₁₀(R)=1, 2, 3, 4, 5. The references sample the exact 50-genotype composite Noncontributor distribution, incurring N=50×R LR computations.

Table 7 shows descriptive log(LR) sampling statistics, along with limiting values for the exact composite distribution. Sampling the genotype set with R=100 references yields a maximum log(LR) of 2.57, suggesting a “zero error” reporting threshold of log(RT)=3 ban. Increased sampling with R=1,000 random profiles finds an log(LR) maximum of 3.15, which would raise the “zero error” log(RT) to 4. Greater R sampling at 10 or 100 thousand DNA profiles produces a maximum log(LR) around 4.5, for a log(RT) of 5 or 6. Hence more comprehensive “zero error” validation testing could cut off LR reporting under the million RT level, declaring all those scores “inconclusive”.

RT cutoff levels rise with more sampling, increasing validation effort and reducing reported DNA information. However, an exact LR false positive error rate can always be computed from an evidence genotype's Noncontributor score distribution (19). Reporting LRs with error rates enables the reporting of all inclusionary LR results, an alternative to RT cutoffs. This ER-based approach eliminates “inconclusive” information loss, providing a frequency context for how often an LR may be misleading. Symmetrically, Contributor distribution FPR error rates enable the reporting of all exclusionary LR results (19).

### 5. Discussion

The first four sections discuss forensic considerations for policy, practice, validation and casework. They address reporting LR error rates, using casework data for genotype validation, and how LR distributions can help defend science and support DNA database hits. The last two sections apply LR distributions to newer forensic DNA assays and medical diagnosis.

### 5.1. Verbal Reporting

Some forensic scientists use verbal equivalents when reporting LR values under a million (60). The chart in Table 8 shows national US guidelines for phrases that convey relative certainty when communicating low LR values. For example, in Johnson one might say an LR of 200 provides Moderate Support for the defendant's inclusion in the DNA mixture.

This qualitative mapping from LR numbers to verbal qualifiers reflects the 1/LR upper bound on LR error rate. However, ER can be exactly computed (19). FIGS. 9 and 12 show that the ER is often much smaller than 1/LR. In Johnson the ER was 1 in 4,100, 20 times less than 1/200. Using the actual ER number is more accurate than stating approximate language and can be more effective in court for conveying LR confidence.

### 5.2. Ground Truth

Some forensic interpretation validation methods require “ground truth” DNA mixtures of known composition (61). However, a genotype can produce a LR distribution that contains all its score-dependent error rate information (19). Combining genotype distributions enables forensic validation on sets of casework items, without “ground truth” knowledge.

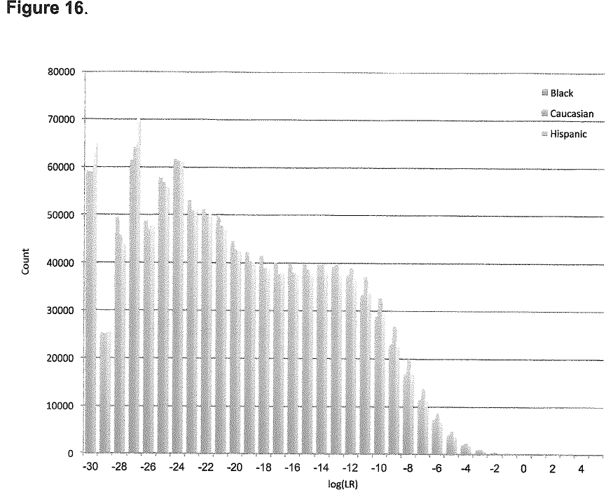

As reported ten years ago (8), “since the [TrueAllele]method's high specificity assures identification hypothesis H with considerable certainty, we can safely examine the Pr{X=x|H} sensitivity distribution of positive log(LR) values.” The Virginia validation study had constructed very accurate Noncontributor distributions from a million LR calculations that compared a hundred mixture casework genotypes with ten thousand randomly sampled population references (FIG. 16). The very low FPR(x) statistically assured the reliability of inclusionary casework match statistics.

Exact composite distributions are readily constructed from evidence genotypes derived from DNA casework items. Since the composites consider all feasible 10²⁴ comparison reference profiles, the complete distributions are far more accurate than sampling just 10⁴ references (FIG. 16). Convolution constructs an exact dense LR score distribution directly from evidence genotypes (19), without sampling references or computing LR values.

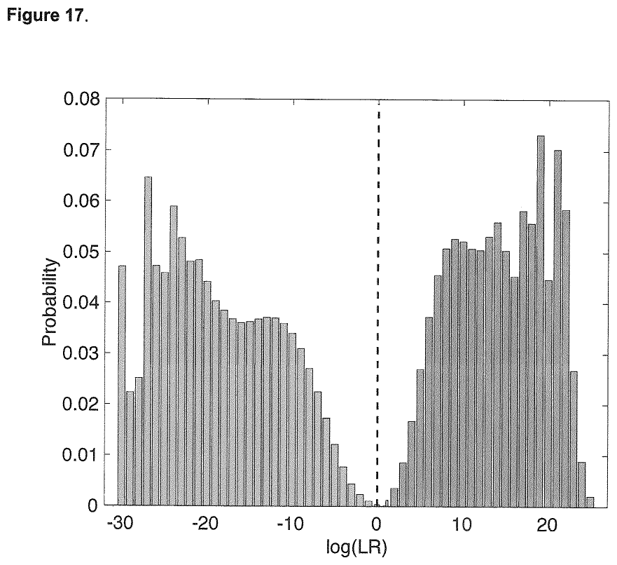

FIG. 17 (left red) shows the exact composite Noncontributor distribution constructed by convolution in under a minute. The original FIG. 16 Virginia validation histogram was developed by laborious profile sampling and LR computation. Comparing FIGS. 16 and 17 (left), the old and new Noncontributor histograms are quite similar. (An older, more conservative, LR calculation method was used in (8). The older method's LR underestimation accounts for the minor histogram differences.)

The double distribution logic symmetrically applies to the reliability of exclusionary casework match statistics. A convolution construction from casework genotypes can form a composite Contributor Y distribution (19) that has high sensitivity. Distribution Y's very low FNR(x) lets us safely examine negative x=log(LR) casework values.

FIG. 17 (right magenta) shows a composite Contributor Y distribution, automatically constructed by convolution from the same validation genotypes. The invention's ROC analysis of score distributions X and Y gives AUC=1, showing a perfect LR classifier. High Contributor Y sensitivity assures a reliable Noncontributor X distribution, just as high Noncontributor X specificity mutually supports the Contributor Y distribution. No “ground truth” is needed for establishing statistical reliability. The error rate information already resides within the Bayesian genotypes derived from empirical STR data.

### 5.3. Defending Science

In United States v. Ravel Mills, TrueAllele reported exclusionary LR values for a defendant. Mills was statistically excluded from a gun (6% component of a 3-person mixture) with log(LR)=−7.86 having log(ER)=−11.18. His exclusionary statistic for a magazine (2% component of a 4-person mixture) was log(LR)=−11.21 having log(ER)=−14.54. The federal prosecutor requested a Daubert hearing to challenge these findings. He claimed that TrueAllele was unreliable for exclusionary LRs, relying on his expert's all-or-none misinterpretation of quantitative LR mixture results from the published Kern County validation study (59).

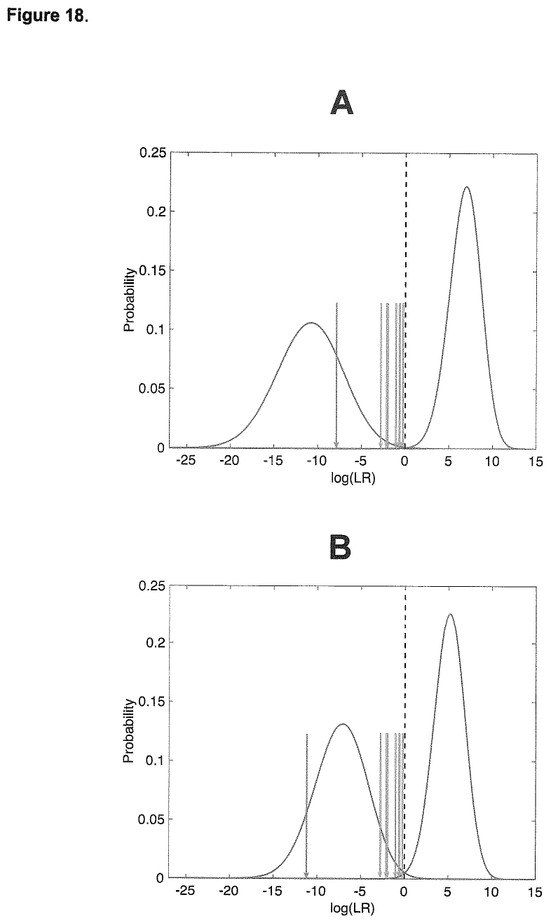

Cybergenetics prepared a scientific response to the prosecution motion (41). This response explained why the opposition argument was based on irrelevant binary mathematics that ignored LR magnitude and didn't account for ER. FIG. 18 shows exact Noncontributor and Contributor distribution pairs (19) for the (A) gun and (B) magazine, with respective AUC values of 1 and 0.9999. The Noncontributor distributions (left blue curves) had informative exclusionary −KL center values of (A) −10.96 and (B) −7.34.

The cited validation study (59) had 7 weak near-zero log(LR) values (red arrows) that were near the evidence Contributor distributions (right blue curves), but far from the defendant's strong log(LR) values (green arrows). As expected, these weak validation exclusionary LRs (recalculated in the relevant software version) had relatively large error rates. But the defendant's strong exclusionary log(LR) values had much smaller error rates. The LR comparison was inapt. The judge rejected the opposition arguments, and accepted TrueAllele as reliable science “on the papers,” without needing to hold a Daubert hearing (62).

### 5.4. Database Search

Early New Year's morning in 2014, a woman was sexually assaulted when walking home through a park at 3 am in Southampton, England. The police collected the victim's vaginal swabs and had them tested for DNA. Searching the DNA profile against England's national DNA database (NDNAD) identified 13 candidate suspects. Non-biological factors pointed to Stuart Burton. But there were 12 other database suspects to assess.

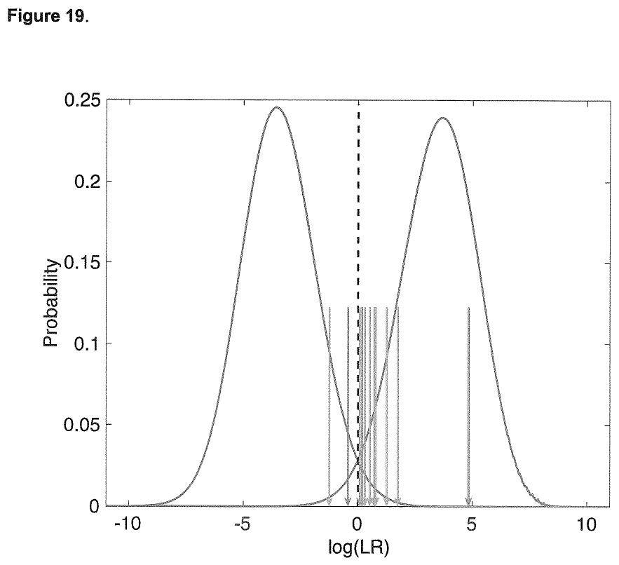

TrueAllele separated the STR data into two components; the major genotype statistically matched the victim. Comparing the minor 15% genotype with Burton gave a log(LR) of 4.83 (FIG. 19, right dark green arrow), which falls within the Contributor distribution (right blue curve). Match statistics were lower to the other 12 NDNAD suspects (green arrows) overlying both distributions (two blue curves) near x=0. The score distribution pair has ROC AUC=0.9980. The log(LR) distance between rightmost Burton and the next largest candidate is over 3 log units.

Error rates can provide additional statistical support for Burton's LR. From the Noncontributor distribution (19), TrueAllele calculated ER's for the (largely inclusionary) LR values, listed in Table 9. The PMEs of the 12 less likely suspects ranged from 1 in 11, to 1 in 808 (last column). Burton's LR error rate was a highly specific 1 in 1.09 million. This ER makes it extremely unlikely that he is a non-contributor whose genotype gave the 67,890 match statistic. Moreover, the invention's high ROC AUC=99.8% established the genotype's high include/exclude classification accuracy. Based in part on these well-founded DNA results, Burton pleaded guilty and was sentenced to twelve years in prison.

### 5.5. Next-Generation Sequencing

Next-generation sequencing (NGS) is a massively parallel DNA testing technology that can simultaneously examine millions of genetic loci. Outside of forensics, NGS has supplanted capillary electrophoresis (CE) assays that are limited to dozens of STR loci. While NGS works differently from CE, both produce quantitative allelic readout data for independent STR loci.

TrueAllele can analyze both CE and NGS data. It forms LR distributions for an evidence genotype, or grouped composites for validation studies (19). A recent TrueAllele validation studied DNA mixtures on NGS (27 loci in Verogen ForenSeq DNA Signature Prep) and CE (21 loci in GlobalFiler) data (63). The laboratory constructed 38 DNA mixtures containing 2 to 4 contributors in varying DNA amounts and mixture weight (MW) percentages. They generated ForenSeq data from the mixtures that were read out on a MiSeq FGx™ NGS instrument (Verogen, San Diego, CA).

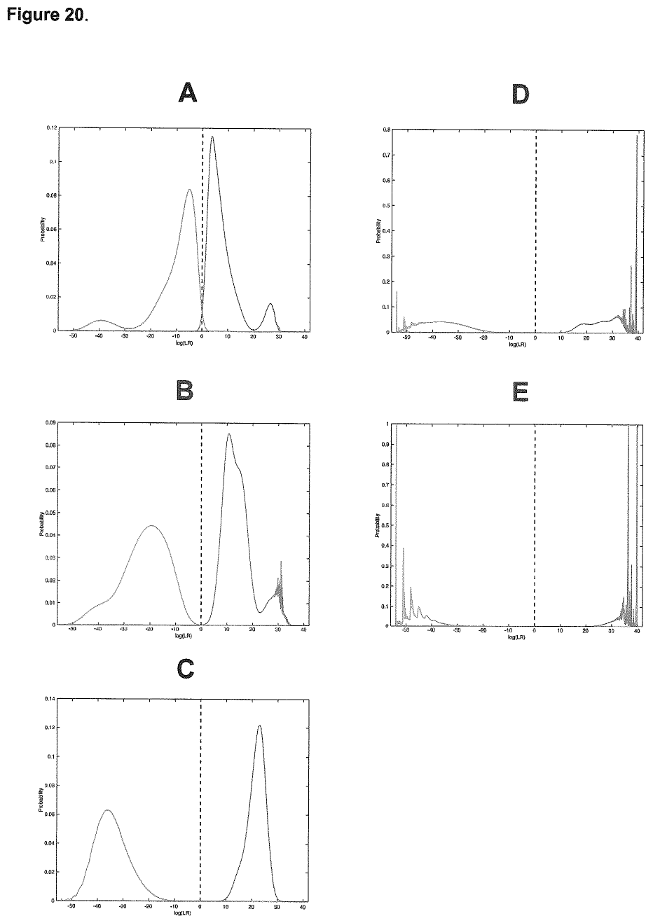

TrueAllele probabilistically unmixed the NGS mixture data to derive 85 separated genotypes. The NGS and CE LR results were concordant at the 20 loci in common. The NGS genotypes were grouped into five quintiles according to their MW, reflecting the relative amount of DNA in the mixture. Composite Noncontributor (red) and Contributor (purple) LR distributions for the five genotype groups are shown in FIG. 20. As MW increases, so too does the average KL information for both noncontributors and contributors, as seen in the greater curve separation. For the 0-20% MW range, AUC=0.9997. In the four MW ranges from 21% to 100% the AUC was 1, showing perfect Contributor-Noncontributor classification.

### 5.6. Medical Diagnosis

Convolved LR distributions have application beyond forensic science. Bayesian reasoning has been used in medicine to diagnose chest pain in the emergency department (ED) and help decide whether a patient should stay in the hospital or go home. Vaizian's BAYES score can reduce unnecessary hospital admissions of healthy ED chest pain patients to 3% (64), down from a 58% false positive rate when using the conventional HEART score.

BAYES forms prior and posterior probability functions for 6 cardiac diagnoses from 25 chest pain clinical variables. For each diagnostic condition, convolving the clinical variable PDFs can rapidly construct a healthy (prior-weighted) or condition (posterior-weighted) LR distribution (19). Comparison of a condition's probabilistic diagnosis with a patient data profile yields a LR. The PME error rate for a diagnosis is calculated by evaluating the cumulative distribution at the patient's log(LR) value.

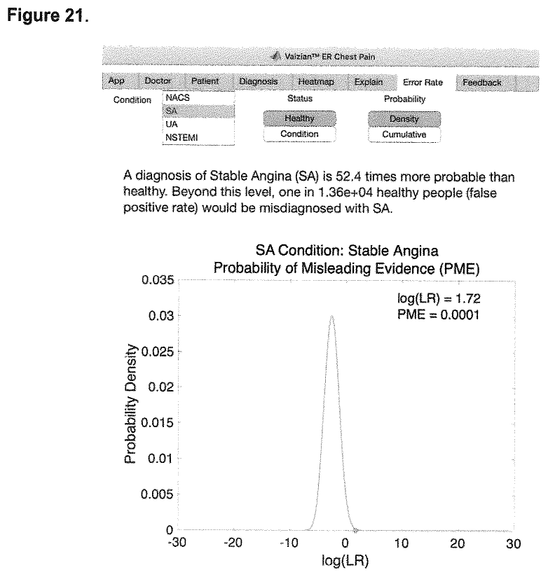

Using the Vaizian web application (48), the LR distribution curve and false positive rate for a Stable Angina diagnosis are shown in FIG. 21. Comparing with a 25-variable clinical profile, the log(LR) value was 1.72 ban. The computer-generated text states the LR value, “A diagnosis of Stable Angina (SA) is 52.4 times more probable than healthy.” The statement continues with the error rate, “Beyond this level, one in 13,600 healthy people (false positive rate) would be misdiagnosed with SA.”

### 6. Conclusion

Science and law require reliable genotype match statistics, along with a measurable error rate. The conditional LR probability distributions, X for Noncontributors and Y for Contributors, inherently reside in the prior and data-derived posterior genotypes. The earlier invention showed how to elicit dense X and Y distributions directly from those uncertain genotypes, as described (19), enabling easy reporting of error rates for LRs. This invention applies those methods and extends them to pairing X and Y for ROC accuracy analysis.

In forensic casework, an evidence contributor genotype is compared with a reference genotype to produce a LR. Previous work gave examples of how evidence-specific X and Y distributions can provide error rates for a LR match statistic (19). Since the mathematics is indifferent to prior or posterior probability labels, the LR can be either inclusionary (using X to find FPR) or exclusionary (using Y for FNR). Low LR values can be safely reported within an error rate's PME frequency context, eliminating reporting threshold “inconclusive” results.

A validation study helps one understand a laboratory process by examining sets of evidence genotypes. A composite X or Y distribution provides a useful statistical summary of laboratory LR behavior for subsets of genotypes sharing similar characteristics. Convolution construction gives exact LR distributions (19), reflecting comparison with all possible 10²⁴ genotypes, not just a sampled 10³⁻⁴ few. Since it is easy to form a Contributor composite (19), laboratory data can also validate the exclusionary LRs and ERs that defendants require.

Data thresholds or human decisions aren't needed to decide which STR peaks to use. A fully Bayesian probability model can input and explain all the data peaks (15). Data are given facts; some likelihood models explain these facts better than others. LR distributions provide the error rates that enable reporting all LR values (19). Different models produce genotype outputs of different log(LR) discriminating ability.

The previously reported X and Y paired LR distributions (19) can be used to measure a genotype's discriminating ability in a new way. Evaluating the genotype's FPR(x) (from X) and TPR(x) (from Y) at every x=log(LR) constructs a continuous ROC curve. The AUC gives the standard Pr{X<Y} accuracy measure. ROC accuracy depends solely on the genotype output, factoring away software input issues like data choices, PG programs, peak height thresholds, and parameter settings. The AUC states how well a genotype's log(LR) comparison score can distinguish between Noncontributor and Contributor. The AUC can objectively compare different genotyping systems and parameters processing the same evidence data.

These findings impact criminal justice. Forensic scientists should be able to use all available DNA data, without artificial restrictions, to help all people all the time. The previous invention showed how to rapidly construct a genotype's exact LR distributions. The distributions produced error rates that assist both prosecution and defense in providing courts with reliable DNA evidence (19). This patent specification showcases many applications of those methods. The specification also introduces a novel way to determine a genotype's discrimination ability and accuracy by combining its two distributions. These objective output measures can help society make better use of DNA evidence.

### Glossary

AUC—ROC measure of classifier accuracy=Pr{X<Y}
Bayes Theorem—Evidence updates belief (q∝λ·p)
Bayesian—Statistical computing based on Bayes Theorem
Category—Contributor or Noncontributor
Complex DNA—An item that has little DNA or is a mixture
Composite distribution—Average of probability distributions
Contributor—Someone who left their DNA
Distribution—Probability of occurrence of possible outcomes
[DNA]—DNA amount or concentration
DNA mixture—An item containing DNA from two or more people
Exclusionary—Match statistic with LR<1, i.e., score log(LR)<0
f_{X}—X probability mass or density function
F_{X}—X cumulative distribution function Pr{X<x}
f_{Y}—Y probability mass or density function
F_{Y}—Y cumulative distribution function Pr{Y<x}
Genotype—Allele pair values at multiple genetic locations
Ground truth—Data assertions believed to be true
Inclusionary—Match statistic with LR>1, i.e., score log(LR)>0
log(LR)—The weight of evidence
LR—Evidential change from prior to posterior probability (q/p)
Monotone—Strictly increasing or decreasing mathematical function
Noncontributor—Someone who did not leave their DNA
Probabilistic genotype—See “Uncertain genotype”
p—Prior genotype probability p(ω) before seeing evidence data
q—Posterior genotype probability q(ω) after seeing evidence data
s—Genotype score s(ω)=log₁₀(q(ω)/p(ω))
Sₓ—An iso-LR genotype subset {ω∈Ω|s(ω)=x}
STRmix™—Probabilistic genotyping software that uses thresholds
TrueAllele®—Bayesian genotyping software that can use all data
Uncertain genotype—A genotype known up to probability
Verbal equivalent—Expresses numerical LR strength in words
x—log(LR) score x-axis coordinate
X—Prior-weighted RV of log(LR) scores
Y—Posterior-weighted RV of log(LR) scores
λ—Likelihood function=Pr{Data|Genotype=ω, other variables}
φ—Standard normal density function
ω—Genotype value in set Ω
Ω—Set of possible multi-locus genotypes

### Abbreviations

AT—Analytical threshold
AUC—Area under the curve
BAYES—Bayesian assessment of your emergency symptoms
CDF—Cumulative distribution function
CE—Capillary electrophoresis
CLT—Central Limit Theorem
DNA—Deoxyribonucleic acid
ED—Emergency department
EPG—Electrophoretogram
ER—Error rate
FBI—Federal Bureau of Investigation
FNR—False negative rate
FPR—False positive rate
H—Hypothesis that someone contributed DNA
HEART—History, ECG, Age, Risk factors and Troponin
HPD—Highest posterior density
KL—Kullback-Leibler divergence
log—Base 10 logarithm
LR—Likelihood ratio
LT-DNA—Low template DNA
MCMC—Markov chain Monte Carlo
NDNAD—The UK's National DNA Database
NGS—Next-generation sequencing
PCR—Polymerase chain reaction
PG—Probabilistic genotyping
PME—Probability of misleading evidence
PMF—Probability mass function
PRS—Positive rate separation
RFU—Relative fluorescent unit
RMP—Random match probability
ROC—Receiver operating characteristic
RT—Reporting threshold
RV—Random variable
STR—Short tandem repeat
TNR—True negative rate
TPR—True positive rate

TABLE 1
The table shows error rate definitions and nomenclature
evaluated at score value x = log(LR). The rows are
divided into negative and positive scores. The columns
are divided into Noncontributor and Contributor categories.

Condition

| | | Noncontributor | Contributor |
|---|---|---|---|
| Score | <x | TNR(x) | FNR(x) |
| | | Pr{X < x} | Pr{Y < x} |
| | | Fₓ(x) | F_{Y}(x) |
| | | Specificity(x) | 1 − Sensitivity(x) |
| | ≥x | FPR(x) | TPR(x) |
| | | Pr{X ≥ x} | Pr{Y ≥ x} |
| | | 1 − Fₓ(x) | 1 − F_{Y}(x) |
| | | 1 − Specificity(x) | Sensitivity(x) |

TABLE 2
The table lists the STR kits and DNA sequencers used
in the study. The numbers of genotypes examined are
shown for these kits and sequencers, partitioned into
inclusionary and exclusionary match statistics.

Number of genotypes

| | inclusionary | exclusionary |
|---|---|---|
| STR kit | | |
| GlobalFiler ™ | 51 | 42 |
| Investigator ® 24plex QS | 3 | 7 |
| PowerPlex ® Fusion | 26 | 18 |
| PowerPlex ® Fusion 6C | 14 | 18 |
| Profiler Plus ® & COfiler ® | 1 | |
| Total | 95 | 85 |
| DNA sequencer (ABI) | | |
| 310 | 1 | |
| 3130 | | 1 |
| 3500 | 74 | 70 |
| 3130xl | 1 | 13 |
| 3500xl | 19 | 1 |
| Total | 95 | 85 |

TABLE 3
This table shows how many contributors were in the
study's DNA items. The number of genotypes corresponding
to these contributor numbers are partitioned by inclusionary
and exclusionary match statistics.

| | |
|---|---|
| Number of | Number of genotypes |

| contributors | inclusionary | exclusionary |
|---|---|---|
| 1 | 5 | 0 |
| 2 | 22 | 22 |
| 3 | 25 | 38 |
| 4 | 21 | 14 |
| 5 | 16 | 9 |
| 6 | 6 | 2 |
| Total | 95 | 85 |

TABLE 4
Noncontributor tail probabilities are shown for inclusionary match
statistics. The rows are binned by positive integer log(LR) values.
The Case part is for the Johnson genotype, while the Validation
part is for a composite distribution formed from 95 low positive
log(LR) genotypes. Within each part the first column is the cumulative
Noncontributor distribution function (CDF), while second column
is the error rate (ER) survival function 1 − CDF.

| | |
|---|---|
| Case | Validation |

| log(LR) | CDF | ER | CDF | ER |
|---|---|---|---|---|
| 0 | 0.992315 | 0.007685 | 0.991899 | 0.008101 |
| 1 | 0.998015 | 0.001985 | 0.997814 | 0.002186 |
| 2 | 0.999584 | 0.000416 | 0.999588 | 0.000412 |
| 3 | 0.999930 | 0.000070 | 0.999945 | 0.000055 |
| 4 | 0.999991 | 0.000009 | 0.999994 | 0.000006 |
| 5 | 0.999999 | 0.000001 | 0.999999 | 0.000001 |

TABLE 5
Contributor tail probabilities are shown for exclusionary match
statistics. The rows are binned by negative integer log(LR)
values. The Case part is for the Robinson genotype, while the
Validation part is for a composite distribution formed from
85 low negative log(LR) genotypes. Each column shows the error
rate (ER) survival function for the log(LR) bin.

| | Case | Validation |
|---|---|---|
| log(LR) | ER | ER |
| 0 | 0.007056 | 0.005171 |
| −1 | 0.001645 | 0.000898 |
| −2 | 0.000315 | 0.000125 |
| −3 | 0.000049 | 0.000015 |
| −4 | 0.000006 | 0.000001 |
| −5 | 0.000001 | 0.000000 |

TABLE 6
The Sandoval data run in TrueAllele, and in STRmix at different
thresholds (RFU column). The table headers show the peak
height threshold (RFU), number of STR loci used (nloci),
number of input data peaks (npeaks), computed mixture weight
(MW), Noncontributor distribution center (−KL), Contributor
distribution center (+KL), ROC accuracy measure (AUC)
and log(LR) comparison match statistic.

| TrueAllele | | | Version 3.25.5840.1 | | | | |
|---|---|---|---|---|---|---|---|
| RFU | nloci | npeaks | MW | −KL | +KL | AUC | log(LR) |
| 0 | 21 | 210 | 0.54 | −11.4206 | 11.0281 | 1.0000 | −6.7828 |

| STRmix | | | Version 2.5.11 | | | | |
|---|---|---|---|---|---|---|---|
| RFU | nloci | npeaks | MW | −KL | +KL | AUC | log(LR) |
| 10 | 19 | 54 | 0.09 | −7.5981 | 4.7557 | 1.0000 | −3.1580 |
| 20 | 18 | 38 | 0.13 | −4.1841 | 2.7046 | 0.9982 | −6.2917 |
| 30 | 16 | 29 | 0.13 | −2.7232 | 1.3926 | 0.9800 | −3.2046 |
| 40 | 14 | 24 | 0.16 | −1.1437 | 0.5802 | 0.8971 | −0.8440 |
| 50 | 12 | 21 | 0.15 | −1.0415 | 0.5232 | 0.8839 | −0.8173 |
| 60 | 10 | 16 | 0.03 | −0.2780 | 0.1460 | 0.7189 | −0.5886 |
| 70 | 9 | 14 | 0.01 | −0.1510 | 0.0684 | 0.6496 | −0.5065 |
| 80 | 9 | 13 | 0.02 | −0.1708 | 0.0904 | 0.6736 | −0.6204 |
| 90 | 8 | 11 | 0.03 | −0.1437 | 0.0655 | 0.6460 | −0.6071 |

TABLE 7
The top rows show statistics for increasing numbers of samples from
the Kern County study Noncontributor distribution, while the bottom
row is for the exact distribution. R is the number of genotype references,
and N is the number of match comparisons. The mean, median, and
maximum summary statistics are shown. The number of positive log(LR)
values, and the percent relative to N are shown.

| R | N | mean | median | maximum | positive | % positive |
|---|---|---|---|---|---|---|
| 10 | 500 | −7.5297 | −7.0133 | 2.5669 | 29 | 5.800 |
| 100 | 5000 | −7.7570 | −7.0009 | 2.5669 | 63 | 1.260 |
| 1000 | 50000 | −8.1047 | −7.3088 | 3.1531 | 440 | 0.880 |
| 10000 | 500000 | −8.2003 | −7.3776 | 4.5458 | 2865 | 0.573 |
| 100000 | 5000000 | −8.1973 | −7.3733 | 4.5843 | 28614 | 0.572 |
| exact | | −8.1747 | −7.3560 | 26.1680 | | 0.638 |

TABLE 8
The SWGDAM Verbal Equivalents table provides verbal
qualifiers for different numerical LR ranges.

| LR for Hₚ Support | |
|---|---|
| and 1/LR for H_{d} | |
| Support | Verbal Qualifier |
| 1 | Uninformative |
| 2-99 | Limited Support |
| 100-9,999 | Moderate Support |
| 10,000-999,999 | Strong Support |
| ≥1,000,000 | Very Strong Support |

TABLE 9
The LR match statistics and PME error probabilities in
Burton, sorted by increasing LR. Each row represents
a different retrieved DNA database genotype, with “SB”
the accused. The last column's “one in” value
is the reciprocal of the PME given in the preceding column.

| Item | LR | log(LR) | PME | one in: |
|---|---|---|---|---|
| 1 | 1/(17.7) | −1.2485 | 0.09155110 | 11 |
| 2 | 1/(2.72) | −0.4339 | 0.03595410 | 28 |
| 3 | 1.21 | 0.0824 | 0.01818210 | 55 |
| 4 | 1.54 | 0.1878 | 0.01569030 | 64 |
| 5 | 2.01 | 0.3025 | 0.01330630 | 75 |
| 6 | 3.35 | 0.5248 | 0.00958381 | 104 |
| 7 | 3.35 | 0.5248 | 0.00958381 | 104 |
| 8 | 5.21 | 0.7166 | 0.00713871 | 140 |
| 9 | 5.90 | 0.7709 | 0.00655932 | 152 |
| 10 | 17.8 | 1.2513 | 0.00297871 | 336 |
| 11 | 17.9 | 1.2535 | 0.00296855 | 337 |
| 12 | 55.6 | 1.7455 | 0.00123809 | 808 |
| SB | 67,890 | 4.8318 | 0.00000092 | 1,087,000 |

The following works are incorporated by reference into this application:

References, all of which are incorporated by reference herein.
1. National Research Council. Evaluation of Forensic DNA Evidence: Update on Evaluating DNA Evidence. Washington, D C: National Academies Press; 1996.
2. Royall R. On the probability of observing misleading evidence. J Am Stat Assoc. 2000; 95(451):760-8.
3. Cover T M, Thomas J A. Elements of information theory. 2nd ed. Hoboken, N.J.: Wiley-Interscience; 2006.
4. Good I J. Probability and the Weighing of Evidence. London: Griffin; 1950.
5. Bieber F R, Buckleton J S, Budowle B, Butler J M, Coble M D. Evaluation of forensic DNA mixture evidence: protocol for evaluation, interpretation, and statistical calculations using the combined probability of inclusion. BMC Genetics. 2016; 17(125):15.
6. Budowle B, Onorato A J, Callaghan T F, Manna A D, Gross A M, Guerrieri R A, et al. Mixture interpretation: defining the relevant features for guidelines for the assessment of mixed DNA profiles in forensic casework. J Forensic Sci. 2009; 54(4):810-21.
7. Perlin M W, Belrose J L, Duceman B W. New York State TrueAllele® Casework Validation Study. J Forensic Sci. 2013; 58(6):1458-66.
8. Perlin M W, Dormer K, Hornyak J, Schiermeier-Wood L, Greenspoon S.
TrueAllele® Casework on Virginia DNA Mixture Evidence: Computer and Manual Interpretation in 72 Reported Criminal Cases. PLoS ONE. 2014; 9(3):e92837.
9. Perlin M W. Inclusion probability for DNA mixtures is a subjective one-sided match statistic unrelated to identification information. J Pathol Inform. 2015; 6(1):59.
10. Scientific Working Group on DNA Analysis Methods (SWGDAM). Short Tandem Repeat (STR) interpretation guidelines. Forensic Sci Commun (FBI). 2000; 2(3).
11. Scientific Working Group on DNA Analysis Methods (SWGDAM).
Interpretation guidelines for autosomal STR typing by forensic DNA testing laboratories. http://www.forensicdna.com/assets/swgdam_2010.pdf: FBI Laboratory; 2010.
12. Perlin M W. Hidden DNA evidence: exonerating the innocent. Forensic Magazine. 2018:10-2.
13. Perlin M W. The Blairsville slaying and the dawn of DNA computing. In: Niapas A, editor. Death Needs Answers: The Cold-Blooded Murder of Dr John Yelenic. New Kensington, P A: Grelin Press; 2013. p. 127-45.
14. Gelman A, Carlin J B, Stem H S, Rubin D. Bayesian Data Analysis. Boca Raton, F L: Chapman & Hall/CRC; 1995.
15. Perlin M W, Sinelnikov A. An Information Gap in DNA Evidence Interpretation. PLoS ONE. 2009; 4(12):e8327.
16. Perlin M W, Legler M M, Spencer C E, Smith J L, Allan W P, Belrose J L, et al. Validating TrueAllele® DNA mixture interpretation. J Forensic Sci. 2011; 56(6):1430-47.
17. Perlin M W. Inclusion probability is a likelihood ratio: implications for DNA mixtures (poster #85). Promega's Twenty First International Symposium on Human Identification; San Antonio, TX2010.
18. Caragine T, Mikulasovich R, Tamariz J, Bajda E, Sebestyen J, Baum H, et al. Validation of testing and interpretation protocols for low template DNA samples using AmpFSTR® Identifiler®. Croat Med J. 2009; 50(3):250-67.
19. Perlin M W. Efficient construction of match strength distributions for uncertain multi-locus genotypes. Heliyon. 2018; 4(10):e00824.
20. Kullback S, Leibler R A. On information and sufficiency. Ann Math Stat. 1951; 22(1):79-86.
21. Fisher R A. Statistical methods for research workers. Edinburgh, London: Oliver and Boyd; 1925.
22. Daubert v. Merrill Dow Pharmaceuticals, Inc.: Supreme Court of the United States; 1993.
23. Hanley J A, McNeil B J. The Meaning and Use of the Area under a Receiver Operating Characteristic (ROC) Curve. Radiology. 1982; 143(1):29-36.
24. Green D M, Swets J A. Signal Detection Theory and Psychophysics. New York: John Wiley & Sons; 1966.
25. Bradley A P. The use of the area under the ROC curve in the evaluation of machine learning algorithms. Pattern Recognition. 1997; 30(7):1145-59.
26. Phillips V L, Saks M J, Peterson J L. The Application of Signal Detection Theory to Decision-Making in Forensic Science. J Forensic Sci. 2001; 46(2):294-308.
27. Riman S, Iyer H, Vallone P M. Examining performance and likelihood ratios for two likelihood ratio systems using the PROVEDIt dataset. PLOS ONE. 2021; 16(9):e0256714.
28. Weber J, May P. Abundant class of human DNA polymorphisms which can be typed using the polymerase chain reaction. American Journal of Human Genetics. 1989; 44:388-96.
29. Mullis K B, Faloona F A, Scharf S J, Saiki R K, Horn G T, Erlich H A. Specific enzymatic amplification of DNA in vitro: the polymerase chain reaction. Cold Spring Harbor Symp Quant Biol. 1986; 51:263-73.
30. Feller W. An Introduction to Probability Theory and Its Applications. Third ed. New York: John Wiley & Sons; 1968.
31. Stolovitzky G, Cecchi G. Efficiency of DNA replication in the polymerase chain reaction. Proc Natl Acad Sci USA. 1996; 93(23):12947-52.
32. Butler J M. Forensic DNA Typing: Biology and Technology Behind STR Markers. New York: Academic Press; 2000.
33. Bayes T, Price R. An essay towards solving a problem in the doctrine of chances. Phil Trans. 1763; 53:370-418.
34. Metropolis N, Rosenbluth A W, Rosenbluth M N, Teller A H, Teller E.
Equation of state calculations by fast computing machines. J Chem Phys. 1953; 21(6):1087-92.
35. Hastings W K. Monte Carlo sampling methods using Markov chains and their applications. Biometrika. 1970; 57(1):97-109.
36. Bauer D W, Butt N, Hornyak J M, Perlin M W. Validating TrueAllele® Interpretation of DNA Mixtures Containing up to Ten Unknown Contributors. J Forensic Sci. 2020; 25(2):380-98.
37. Billingsley P. Probability and Measure. Third ed. New York: John Wiley & Sons; 1995.
38. O'Hagan A, Forster J. Bayesian Inference. Second ed. New York: John Wiley & Sons; 2004.
39. Feller W. On a general class of “contagious” distributions. The Annals of Mathematical Statistics. 1943; 14(4):389-99.
40. Buckleton J S, Pugh S N, Bright J A, Taylor D A, Curran J M, Kruijver M, et al. Are low LRs reliable? Forensic Sci Int Genet. 2020; 49:102350.
41. Perlin M W. Expert Declaration (U S v. Mills). Pittsburgh, P A; 2023 July 20.
42. Metz C E. Basic Principles of ROC Analysis. Seminars in Nuclear Medicine. 1978; VIII(4):283-98.
43. Bamber D. The area above the ordinal dominance graph and the area below the receiver operating characteristic graph. Journal of Mathematical Psychology. 1973; 12(4):387-415.
44. Cali C, Longobardi M. Some mathematical properties of the ROC curve and their applications. Ricerche di Matematica. 2015; 64:391-402.
45. Youden W J. Index for rating diagnostic tests. Cancer Research. 1950; 3(1):32-5.
46. Hwang Y-T, Hung Y-H, Wang C C, Terng H-J. Finding the Optimal Threshold of a Parametric ROC Curve Under a Continuous Diagnostic Measurement. Revstat—Statistical Journal. 2018; 16(1):23-43.
47. Kumar R, Indrayan A. Receiver operating characteristic (ROC) curve for medical researchers. Indian Pediatrics. 2011; 48(4):277-87.
48. Vaizian. Vaizian™ E R Chest Pain 2022 [Available from: https://heartbeat.vaizian.com/webapps/home/session.html?app=HeartBeatWebApp.
49. Perlin M W, Butt N, Wilson M R. Commentary on: Thompson W C. Uncertainty in probabilistic genotyping of low template DNA: A case study comparing STRmix and TrueAllele®. J Forensic Sci. 2023; 68(3):1049-63. J Forensic Sci. 2024.
50. Perlin M W. California baggie DNA mixture evidence. 1 ed. SSRN: Mendeley Data Repository; 2023.
51. Perlin M W, Allan W P, Bracamontes J M, Danser K R, Legler M M. Reporting exclusionary results on complex DNA evidence, with a response to ‘Uncertainty in probabilistic genotyping of low template DNA: A case study comparing STRmix™ and TrueAllele®’ software [Preprint]. 2023 [updated May 18, 2023. Available from: https:/ssrn.com/abstract=4449313.
52. Perlin M W. NIJ40 DNA mixture files. In: Cybergenetics, editor. 1 ed. PLoS ONE: Mendeley Data Repository; 2018.
53. Lilliefors H W. On the Kolmogorov-Smirnov Test for Normality with Mean and Variance Unknown. Journal of the American Statistical Association. 1967; 62(318):399-402.
54. Chernoff H. A Measure of Asymptotic Efficiency for Tests of a Hypothesis Based on the sum of Observations. The Annals of Mathematical Statistics. 1952; 23(4):493-507, 15.
55. Gordon R D. Values of Mills' Ratio of Area to Bounding Ordinate and of the Normal Probability Integral for Large Values of the Argument. The Annals of Mathematical Statistics. 1941; 12(3):364-6, 3.
56. Possley M. Kerry Robinson Michigan: University of Michigan; 2020 [Available from: https://www.law.umich.edu/special/exoneration/Pages/casedetail. aspx?caseid=5671.
57. Noel S, Noel J, Granger D, Lefebvre J-F, Seguin D. STRmix™ put to the test: 300 000 non-contributor profiles compared to four-contributor DNA mixtures and the impact of replicates. Forensic Science International: Genetics. 2019; 41:24-31.
58. McCarthy-Allen M, Bleka Ø, Ypma R, Gill P, Benschop C. ‘Low’ LRs obtained from DNA mixtures: On calibration and discrimination performance of probabilistic genotyping software. Forensic Science International: Genetics. 2024; 73:103099.
59. Perlin M W, Hornyak J, Sugimoto G, Miller K. TrueAllele® Genotype Identification on DNA Mixtures Containing up to Five Unknown Contributors. J Forensic Sci. 2015; 60(4):857-68.
60. SWGDAM. Recommendations of the SWGDAM ad hoc working group on genotyping results reported as likelihood ratios. In: Justice, editor. Washington, D C: FBI 2018.
61. Adamowicz M S, Rambo T N, Clarke J L. Internal Validation of MaSTR™ Probabilistic Genotyping Software for the Interpretation of 2-5 Person Mixed DNA Profiles. Genes. 2022; 13(8):1429.
62. United States v. Ravel Mills. Superior Court of the District of Columbia; 2023.
63. Legler M, Bracamontes J M, Van Buren M. TrueAllele® Casework validation of the Verogen ForenSeq DNA Signature Prep Kit Primer Set B and the MiSeq FGX. Thirty Fourth International Symposium on Human Identification; Denver, CO: Promega; 2023.
64. Perlin M W, Accilien Y-D. Bayesian intelligence for medical diagnosis: a pilot study on patient disposition for emergency medicine chest pain. Diagnosis. 2024.

Although the invention has been described in detail in the foregoing embodiments for the purpose of illustration, it is to be understood that such detail is solely for that purpose and that variations can be made therein by those skilled in the art without departing from the spirit and scope of the invention except as it may be described by the following claims.

1. A method of convicting a person guilty of a crime comprised of the steps:
a. collecting evidence from a scene where a crime has occurred that contains DNA from a specific person;
b. processing the evidence to produce DNA data;
c. interpreting the data to infer a probabilistic evidence genotype;
d. deriving from the evidence genotype a first score distribution that describes the frequency of scores from reference genotypes of people who did not contribute their DNA to the evidence;
e. deriving a second score distribution from the evidence genotype that describes the frequency of scores from reference genotypes that are probabilistically similar to the genotype of the person who contributed their DNA to the evidence;
f. constructing a curve from pairs of error rates at a plurality of scores, where each error rate pair comes from the first and second distributions evaluated at the score;
g. finding an area under the curve;
h. using the area as a measure of the score's ability to accurately discriminate between people who did or did not leave their DNA in the evidence;
i. organizing the genotype's score distribution pair, curve, and area into a discrimination package;
j. comparing the evidence genotype with the person's genotype to produce an inclusionary score;
k. presenting the score, together with a component of the package, in a legal proceeding;
l. convicting the person of the crime, based on the score presentation; and
m. incarcerating the person.

2. A method as described in claim 1 where before the step of presenting in a legal proceeding, there is the additional step of using the score and a component of the package to persuade a judge to admit the DNA evidence.

3. A method as described in claim 1 where the score is a likelihood ratio.

4. A method as described in claim 1 where only one genotype inferred from the evidence data is used in deriving the first and second score distributions.

5. A method as described in claim 1 where steps a through i are completed before comparing the evidence genotype with a reference genotype.

6. A method of acquitting a person not guilty of a crime comprised of the steps:
a. collecting evidence from a scene where a crime has occurred that does not contain DNA from a specific person;
b. processing the evidence to produce DNA data;
c. interpreting the data to infer a probabilistic evidence genotype;
d. deriving from the evidence genotype a first score distribution that describes the frequency of scores from reference genotypes of people who did not contribute their DNA to the evidence;
e. deriving a second score distribution from the evidence genotype that describes the frequency of scores from reference genotypes that are probabilistically similar to the genotype of someone who is not the person and contributed their DNA to the evidence;
f. constructing a curve from pairs of error rates at a plurality of scores, where each error rate pair comes from the first and second distributions evaluated at the score;
g. finding an area under the curve;
h. using the area as a measure of the score's ability to accurately discriminate between people who did or did not leave their DNA in the evidence;
i. organizing the genotype's score distribution pair, curve, and area into a discrimination package;
j. comparing the evidence genotype with the person's genotype to produce an exclusionary score;
k. presenting the score, together with a component of the package, in a legal proceeding;
l. acquitting or exonerating the person of the crime, based on the score presentation; and
m. releasing the person.

7. A method as described in claim 6 where before the step of presenting in a legal proceeding, there is the additional step of using the score and a component of the package to persuade a judge to admit the DNA evidence.

8. A method as described in claim 6 where the score is a likelihood ratio.

9. A method as described in claim 6 where only one genotype inferred from the evidence data is used in deriving the first and second score distributions.

10. A method as described in claim 6 where steps a through i are completed before comparing the evidence genotype with a reference genotype.

11. A non-transitory readable storage medium includes a computer program stored on the storage medium for convicting a person guilty of a crime having the computer-generated steps of:
a. processing evidence from a scene where a crime has occurred that contains DNA from a specific person to produce DNA data;
b. interpreting the data to infer a probabilistic evidence genotype;
c. deriving from the evidence genotype a first score distribution that describes the frequency of scores from reference genotypes of people who did not contribute their DNA to the evidence;
d. deriving a second score distribution from the evidence genotype that describes the frequency of scores from reference genotypes that are probabilistically similar to the genotype of the person who contributed their DNA to the evidence;
e. constructing a curve from pairs of error rates at a plurality of scores, where each error rate pair comes from the first and second distributions evaluated at the score;
f. finding an area under the curve;
g. using the area as a measure of the score's ability to accurately discriminate between people who did or did not leave their DNA in the evidence;
h. organizing the genotype's score distribution pair, curve, and area into a discrimination package;
i. comparing the evidence genotype with the person's genotype to produce an inclusionary score; wherein the score is presented, together with a component of the package, in a legal proceeding causing conviction of the person of the crime, based on the score presentation, and incarcerating the person.
